(19) United States
(12) Patent Application Publication
(10) Pub. No.: US 2025/0325657 A1
(43) Pub. Date: Oct. 23, 2025

(54) MODIFIED VACCINIA ANKARA (MVA) VACCINE

(71) Applicant: CITY OF HOPE, Duarte, CA (US)

(72) Inventors: Javier Gordon OGEMBO, San Dimas, CA (US); Gabriela Martinez ESCALANTE, Duarte, CA (US); Ivana Gabriela REIDEL, Covina, CA (US)

(73) Assignee: CITY OF HOPE, Duarte, CA (US)

(21) Appl. No.: 19/183,670

(22) Filed: Apr. 18, 2025

Related U.S. Application Data
(60) Provisional application No. 63/636,060, filed on Apr. 18, 2024.

(51) Int. Cl. A61K 39/295; A61K 39/00; A61K 39/285; A61P 31/22

(52) U.S. Cl. CPC A61K 39/295; A61K 39/285; A61P 31/22; A61K 2039/5256; A61K 2039/545; A61K 2039/572; A61K 2039/575

(57) ABSTRACT

Reconstructed Modified Vaccinia Ankara (rMVA) vectors configured to encode stable Epstein Bar Virus (EBV) glycoproteins gp42, gL, gH, gp350, and gB, compositions comprising the rMVA vectors, vaccines comprising the compositions, and methods of preventing EBV infection by administering the vaccines and eliciting an innate and humoral immune response.

### CROSS-REFERENCE TO RELATED APPLICATIONS

This application claims the benefit of U.S. Provisional Patent Application No. 63/636,060, filed on Apr. 18, 2024, the contents of which is incorporated by reference in its entirety.

### STATEMENT REGARDING FEDERALLY SPONSORED RESEARCH

This invention was made with government support under R56 Al148295, awarded by the National Institutes of Health; and W81XWH-20-1-0401, awarded by the U.S. Army Medical Research and Development Command. The government has certain rights in the invention.

### TECHNICAL FIELD

Epstein-Barr virus vaccines.

### BACKGROUND

Epstein-Barr virus (EBV) is a gamma-herpesvirus prevalent in >90% of the human population¹. As the first oncogenic virus to be identified, it is associated with about 350,000 new cases of epithelial and lymphoid malignancies every year, and is also the causative agent of infectious mononucleosis^{1,2}. EBV is also associated with several autoimmune diseases and was recently established as a major causative factor in the development of multiple sclerosis³⁻⁵. Despite four decades of EBV research since its discovery, there remains a need for improved prophylactic vaccines against the virus or its associated diseases⁶.

### BRIEF DESCRIPTION OF THE DRAWINGS

FIGS. 1A-1E show the generation and characterization of MVA-EBV5-2. FIG. 1A shows a schematic representation of MVA-EBV5-2 virus, with multivalent glycoprotein expression cassettes incorporated in the 69R/70L (G1L, gp350-gB) and 64L/65L (IGR3, gp42-gL-gH) MVA genomic sites. FIG. 1B shows a diagram depicting the reconstitution of recombinant MVA in BHK-21 cells, followed by viral serial passaging to evaluate transgene stability. FIG. 1C shows PCR amplification of the two glycoprotein expression cassettes in MVA-EBV5-2, illustrated in FIG. 1A, using DNA from uninfected BHK-21 cells (BHK), BHK-21 cells infected with empty MVA (BHK-MVA), or BHK-21 cells infected with serially passaged MVA-EBV5-2 virus (p0, p4, p7, and p10). NC denotes negative control, no DNA. Expected band sizes are listed above each gel and are indicated with arrows. FIG. 1D shows binding of anti-gp350 antibody HB5, anti-gp42 antibody 15C8, and anti-B5R hybridoma supernatant to total protein from uninfected BHK-21 cells, BHK-21 cells infected with empty MVA, or BHK-21 cells infected with serially passaged MVA-EBV5-2 virus (p0-p10) was assessed using immunoblot. Bands corresponding to gp350, gp42, and B5R are indicated with arrows. B5R is a vaccinia antigen, and is used as a loading control. FIG. 1E shows surface binding of the anti-gp350mAb HB5, anti-gB mAb AMMO5, anti-gp42 mAb F-2-1, anti-gL mAb E1D1, and anti-gH mAb CL40 to BHK-21 cells infected with serially passaged MVA-EBV5-2 virus (p0, p4, p7 and p10) was measured by flow cytometry analysis. Each bar represents the mean percent (%) +SD of infected cells from triplicate infections with positive signal for binding.

FIGS. 2A-2E show immunogenicity of MVA-EBV5-2 in female BALB/c mice. FIG. 2A shows that female BALB/c mice were immunized with the indicated treatments (inset table, right) on Day 0, 28, and 56 (n=8/group). Blood was collected at the indicated timepoints (red droplets), and mice were terminally bled on Day 182. FIG. 2B shows that IgG binding titers to gp350, gB, and gp42gHgL were measured using ELISA in pooled mouse serum ( 1/900 dilution). Each dot represents the mean of triplicate measurements, and black arrows represent immunization timepoints. FIG. 2C shows that IgG1 binding titers to EBV gp350, gB and gp42gHgL were measured by ELISA in individual mouse serum samples on Day 49 and 84 post-immunization ( 1/900 dilution). Each dot represents the mean of duplicate measurements for each animal, with the median and interquartile range shown for each group at each timepoint. Statistical differences were determined using Tukey's multiple comparison test (*=p<0.05, **=p<0.01, ***=p<0.001, ****=p<0.0001). FIG. 2D shows that IgG2a binding titers to EBV gp350, gB and gp42gHgL were measured by ELISA in individual mouse serum samples on Day 49 and 84 post-immunization (1/900 dilution). Each dot represents the mean of duplicate measurements for each animal, with the median and interquartile range shown for each group at each timepoint. Statistical differences in were determined using Tukey's multiple comparison test (*=p<0.05, **=p<0.01, ***=p<0.001, ****=p<0.0001). FIG. 2E shows the ability of Day 49 and 84 pooled sera to neutralize EBV infection in HEK-293 (left) and Raji (right) cells was measured via in vitro neutralization assays. Serum titration curves for each treatment group (% neutralization) are shown for each cell line, with the top dotted line representing 80% neutralization (IC80) and the bottom dotted line representing 50% neutralization (IC50).

FIGS. 3A-3E show immunogenicity of MVA-EBV5-2 in male BALB/c mice. FIG. 3A shows that male BALB/c mice were immunized with the indicated treatments on Day 0, 21 and 56 (n=8/group). Blood was collected at the indicated timepoints, and mice were terminally bled on Day 119. FIG. 3B shows that IgG binding titers to EBV gp350, gB, and gp42gHgL were measured using ELISA in pooled mouse serum (1/900 dilution). Each dot represents the mean of triplicate measurements, and black arrows represent immunization timepoints. FIG. 3C shows that IgG1 binding titers to EBV gp350, gB and gp42gHgL were measured by ELISA in individual mouse serum samples on Day 49 and 84 post-immunization ( 1/900 dilution). Each dot represents the mean of duplicate measurements for each animal, with the median and interquartile range shown for each group at each timepoint. Statistical differences in were determined using Tukey's multiple comparison test (*=p<0.05, **=p<0.01, ***=p<0.001, ****=p<0.0001). FIG. 3D shows that IgG2a binding titers to EBV gp350, gB and gp42gHgL were measured by ELISA in individual mouse serum samples on Day 49 and 84 post-immunization ( 1/900 dilution). Each dot represents the mean of duplicate measurements for each animal, with the median and interquartile range shown for each group at each timepoint. Statistical differences were determined using Tukey's multiple comparison test (*=p<0.05, **=p<0.01, ***=p<0.001, ****=p<0.0001). FIG. 3E shows the ability of Day 49 and 84 pooled sera to neutralize EBV infection in HEK-293 (left) and Raji (right) cells was measured via in vitro neutralization assays. Serum titration curves for each treatment group (% neutralization) are shown for each cell line, with the top dotted line representing 80% neutralization (IC80) and the bottom dotted line representing 50% neutralization (IC50).

FIGS. 4A-4D show experimental rhesus macaque information and rhLCV screening. FIG. 4A shows a table describing individual rhesus macaque demographic information and distribution into treatment groups. FIG. 4B shows that IgG binding titers to rhLCV gp350 were measured by ELISA in individual macaque serum samples prior to study start (Day-7, FIG. 5), ( 1/100). Each bar represents the mean of duplicate measurements for each individual animal. FIG. 4C shows SDS-PAGE/Coomassie stain analysis of purified rhLCV-gp350 used in ELISA described in FIG. 4B. R: reducing conditions; NR: non-reducing conditions. Bands corresponding to rhLCV gp350 are indicated with arrows. FIG. 4D shows binding of monoclonal Anti-His antibody (mAb, left) and polyclonal serum from a rhesus macaque infected with rhLCV (pAb, right) to purified rhLCV-gp350 protein used in ELISA described in FIG. 4B was assessed using immunoblot. Bands corresponding to rhLCV gp350 are indicated with arrows.

FIGS. 5A-5E show immunogenicity of MVA-EBV5-2 in rhLCV-negative rhesus macaques. FIG. 5A shows that rhLCV-negative rhesus macaques were immunized with the indicated treatments on Day 0 and Day 28 (n=5/group). Blood and saliva were collected on the indicated timepoints. FIG. 5B shows that IgG binding titers to EBV gp350, gB, and gp42gHgL were measured by ELISA in individual macaque serum samples (1/100 dilution). The median and interquartile range are shown for each group at each timepoint, each animal was tested in duplicate. Black arrows represent immunization timepoints. FIG. 5C shows that IgG binding titers to EBV gp350, gB, and gp42gHgL were measured by ELISA in individual macaque serum samples ( 1/100 dilution). This data is the same as presented in FIG. 5B, but shown here for individual animals, each dot representing the mean of duplicate measurements for each animal, with the median and interquartile range shown for each group at each timepoint. Statistical differences were determined using Tukey's multiple comparison test (*=p<0.05, **=p<0.01). FIG. 5D shows that IgG binding titers to EBV gp350, gB, and gp42gHgL were measured by ELISA in individual macaque saliva samples (undiluted). The median and interquartile range are shown for each group at each timepoint, each animal was tested in duplicate. FIG. 5E shows that IgG binding titers to EBV gp350, gB, and gp42gHgL were measured by ELISA in individual macaque saliva samples (undiluted). This data is the same as presented in FIG. 5D, but shown here for individual animals, each dot representing the mean of duplicate measurements for each animal, with the median and interquartile range shown for each group at each timepoint. Statistical differences were determined using Tukey's multiple comparison test (*=p<0.05, **=p<0.01).

FIGS. 6A-6F show neutralizing activity in the serum of MVA-EBV5-2-immunized BALB/c rhesus macaques. The ability of serially-diluted Day 56 individual animal serum from rhesus macaques in FIG. 5 to neutralize EBV infection in HEK-293 (FIG. 6A) and Raji (FIG. 6B) cells was measured via in vitro neutralization assay. Each dot represents the last dilution at which ≥50% (left panel) or ≥80% (right panel) neutralization was achieved for each animal (IC50 and IC80, respectively), as compared to the level of infection in the presence of Day −7 serum for each corresponding animal. The median and interquartile range is shown for each group. Statistical differences were determined using Kruskal-Wallis test (*=p<0.05). FIG. 6C-6D show neutralization titration curves for immunized rhesus macaque serum. The ability of serially diluted Day 56 sera from individual rhesus macaques in FIGS. 6A-6B to neutralize EBV infection was measured via in vitro neutralization assays. Individual animal titration curves for each treatment group (% neutralization) from which the FIG. 6 IC50 and IC80 plots were derived are shown for HEK-293 (FIG. 6C) and Raji (FIG. 6D) cells. Samples that did not achieve neutralization as compared to Pre-immune serum were assigned a value of 0%. Top dotted line represents 80% neutralization; bottom dotted line represents 50% neutralization. FIGS. 6E-6F show MVA-EBV5-2-immune rhesus macaque serum glycoprotein-specific antibody depletion. FIG. 6E shows that the ability of Day 56 pooled sera from rhesus macaques in FIG. 6A-B to neutralize EBV infection before and after anti-gp350 antibody depletion was measured via in vitro neutralization assays. Pooled sera at a 1/12.5 dilution from each treatment group was incubated with a gp350-coated nitrocellulose membrane, and before neutralization assays, IgG binding levels to EBV gp350 (1/50 sera dilution) were measured using ELISA (first panel) in depleted sera (gp350), sera incubated with a BSA-blocked nitrocellulose membrane (mock), or undepleted sera (none); bar graphs represent the mean+SD of duplicate measurements for each group. Anti-gp350 antibody-depleted sera and control sera was subsequently used in HEK-293 (middle panel) and Raji (right panel) cell neutralization assays; bar graphs represent the mean+SD neutralization of triplicate measurements for each group, at a 1/50 sera dilution. FIG. 6F shows that the ability of Day 56 MVA-EBV5-2 pooled sera from rhesus macaques in FIG. 6A-B to neutralize EBV infection before and after anti-gp350,-gB, and-gp42gHgL antibody depletion was measured via in vitro neutralization assays. Pooled sera at a 1/12.5 dilution from each treatment group was incubated with gp350-, gB-, or gp42gHgL-coated nitrocellulose membranes, and before neutralization assays, IgG binding levels to each glycoprotein/glycoprotein complex ( 1/50 sera dilution) were measured using ELISA (top panel) in glycoprotein-depleted sera, sera incubated with a BSA-blocked nitrocellulose membrane (mock), or undepleted sera (none); bar graphs represent the mean+SD of duplicate measurements for each group. Glycoprotein-specific antibody-depleted sera and control sera was subsequently used in HEK-293 and Raji (bottom panel) cell neutralization assays; bar graphs represent the mean+SD neutralization of triplicate measurements for each group, at a 1/50 sera dilution.

FIGS. 7A-7G show that MVA-EBV5-2-elicited antibodies protect NSG huMice from low-dose EBV challenge better than antibodies elicited by a monovalent gp350 vaccine. FIG. 7A shows that female and male NSG huMice were passively immunized intraperitoneally (i.p.) with 500 μl of Day 56 MVA-EBV5-2-immune sera, Day 56 MVA-gp350-immune sera, or Pre-immune sera from rhesus macaques (n=7/group). Twelve hours post-immunization, mice were challenged with 5×10³ Raji IU Akata-EBV-eGFP i.p., then monitored for 56 days, after which animals were euthanized and tissues were collected. An additional group of mice (n=2) were neither immunized nor challenged to serve as a Sham control group FIG. 7B shows that the percent (%) hCD45+ lymphocytes was assessed using flow cytometry in collected circulating blood of NSG mice reconstituted with hCD34+ cells, 12 weeks post-engraftment and prior to immunization. Shown are individual mouse measurements after allocation into treatment groups using a balanced allocation logarithm. Note that the two mice in the Sham group had low percentages of hCD45+ lymphocytes, and were not included in the balanced allocation algorithm to prioritize NSG huMice with high levels of hCD45+ lymphocytes in experimental groups. The median and interquartile range are shown for each group. Statistical differences were determined using Tukey's multiple comparison test (ns=not significant, ****=p<0.0001). Viral DNA in the peripheral blood (FIG. 7C) or spleens (FIG. 7D) of NSG huMice was quantified via qPCR after euthanization. Each dot represents the mean of individual mouse measurements, each animal was tested in duplicate. The median and interquartile range are shown for each group. The dashed line represents the limit of detection. Statistical differences were determined using Mann-Whitney test (*=p<0.05). FIG. 7E shows that the presence of EBER in the spleens of NSG huMice was assessed via in situ hybridization after euthanization. Shown is the EBER spleen staining score for each mouse on a grading scale of 0-4. Crossed-out rectangles represent non-evaluable mice that succumbed before sample collection. Perimeter (FIG. 7F) and area (FIG. 7G) of collected spleens from NSG huMice were quantified using IC Measure software. Shown are individual mouse measurements with the median and interquartile range shown for each group. Statistical differences were assessed using Kruskal-Wallis test, but no differences were detected.

FIGS. 8A-8G show that MVA-EBV5-2-elicited antibodies protect NSG huMice from high-dose EBV challenge better than antibodies elicited by a monovalent gp350 vaccine. FIG. 8A shows that female and male NSG huMice were passively immunized intraperitoneally (i.p.) with 500 μl of Day 56 MVA-EBV5-2-immune sera, Day 56 MVA-gp350-immune sera, or Pre-immune sera from rhesus macaques (n=7/group). Twelve hours post-immunization, mice were challenged with 5×10⁴ Raji IU Akata-EBV-eGFP i.p., then monitored for 28 days, after which animals were euthanized and tissues were collected. An additional group of mice (n=6) were neither immunized nor challenged to serve as a Sham control group. FIG. 8B shows that percent (%) hCD45+ lymphocytes was assessed using flow cytometry in collected circulating blood of NSG mice reconstituted with hCD34+ cells, 12 weeks post-engraftment and prior to immunization. Shown are individual mouse measurements after allocation intro indicated treatment groups using a balanced allocation logarithm. The median and interquartile range are shown for each group. Statistical differences were determined using Tukey's multiple comparison test (ns=not significant). Viral DNA in the peripheral blood (FIG. 8C) or spleens (FIG. 8D) of NSG huMice was quantified via qPCR after euthanization. Each dot represents the mean of individual mouse measurements, each animal was tested in duplicate. The median and interquartile range are shown for each group. The dashed line represents the limit of detection. Statistical differences were determined using Mann-Whitney test (ns=not significant, *=p<0.05, **=p<0.01). FIG. 8E shows that the presence of EBER in the spleens of NSG huMice was assessed via in situ hybridization after sacrifice. Shown is the EBER spleen staining score for each mouse on a grading scale of 0-4. Crossed-out rectangles represent non-evaluable mice that succumbed before sample collection. Perimeter (FIG. 8F) and area (FIG. 8G) of collected spleens from NSG huMice were quantified using IC Measure software. Shown are individual mouse measurements with the median and interquartile range shown for each group. Statistical differences were assessed using Kruskal-Wallis test, but no differences were detected.

FIG. 9A-9C shows the assessment of human lymphocyte engraftment in NSG mice, and kinetics of UV-EBV-immune RM sera IgG in NSG huMice. FIG. 9A shows that percent human CD45 (hCD45)+lymphocytes was assessed by flow cytometry in the circulating blood of CD34+ cell-engrafted NSG mice from FIG. 7 and Table 1 and FIG. 8 Table 2. Shown is the gating strategy used in the assessment. FIG. 9B shows that NSG huMice were passively immunized with 500 μl of Day 56 UV-EBV-immune sera from rhesus macaques in FIG. 5A-5E to assess the kinetics of glycoprotein-specific rhesus IgG (n=3/timepoint). Mice were sequentially sacrificed at −1-, 3-, 6-, 12- and 24-hours post-immunization for blood collection, and the intraperitoneal cavity was examined for the presence of rhesus macaque sera. IgG binding titers to EBV gp350 (FIG. 9C) were measured by ELISA in individual NSG huMouse serum samples from (FIG. 9B) ( 1/50 dilution). Each dot represents the mean of individual animal measurements in each group per timepoint, each animal tested in triplicate. The line passes through the mean of each timepoint.

FIG. 10 shows EBER-ish staining of spleens from NSG huMice in low-dose EBV challenge study. EBER-ish spleen staining slides for humanized mice described in FIG. 7 and Table 1. Shown are slides for a representative control animal (Sham, #3), and all seven EBER-positive spleens in the study with corresponding treatment group and animal #indicated. Scale: black bar denotes 500 μm in each main panel; black bar denotes 100 μm in each zoomed-in panel.

FIG. 11 shows EBER-ish staining of spleens from NSG huMice in high-dose EBV challenge study. EBER-ish spleen staining slides for humanized mice described in FIG. 8 and Table 2. Shown are slides for a representative control animal (Sham, #5), and all six EBER-positive spleens in the study with corresponding treatment group and animal #indicated. Scale: black bar denotes 500 μm in each main panel; black bar denotes 100 μm in each zoomed-in panel.

FIGS. 12A-12B show survival and comparison of spleen EBER-ish staining scores against BALF5 copies/μg of DNA in humanized mouse EBV challenge studies.

FIG. 12A shows the comparison of EBER-ish staining scores against BALF5 copies/μg of DNA in individual mouse spleens from humanized mice in both low (FIG. 7 and Table 1) and high (FIG. 8 and Table 2) dose challenge studies. FIG. 12B shows survival curves for NSG huMice in low (left, FIG. 7) and high (right, FIG. 8) EBV dose challenge studies. Statistical differences were determined using Log-rank test, but the only difference detected was between the Sham and MVA-gp350 groups in the high dose study (p=0.0347).

FIGS. 13A-13E show Preliminary characterization of recombinant MVA vectors incorporating EBV glycoproteins. FIG. 13A shows schematic representations of MVA-EBV5-1, MVA-EBV5-5, MVA-EBV-gp350-gB, and MVA-EBV-gp42gLgH with EBV glycoprotein expression cassettes incorporated in the indicated MVA genomic sites. FIG. 13B shows that surface binding of the anti-gp350 monoclonal antibody (mAb) HB5, anti-gB mAb AMMO5, anti-gp42 mAb F-2-1, anti-gL mAb E1D1, and anti-gH mAb CL40 to BHK-21 cells infected with the indicated MVA vectors was measured using flow cytometry. Each bar represents the mean percent (%)+SD of infected cells from duplicate or triplicate infections with positive signal for binding. FIG. 13C shows validation of the surface binding of the indicated glycoprotein-specific monoclonal antibodies (mAbs) used for flow cytometry evaluation of MVA-EBV5-2. BHK-21 cells infected with empty MVA virus or BHK-21 cells infected with MVA-EBV5-2 virus were incubated with anti-gp350 mAb HB5, anti-gp42 mAb F-2-1, anti-gL mAb E1D1, anti-gH mAb CL40, and anti-gB mAb AMMO5. The antigen-antibody interaction was detected using a secondary antibody conjugated to Alexa Fluor 647 (AF-647) and measured using flow cytometry. Each bar represents the mean percent (%)+SD of infected cells from triplicate infections with positive signal for binding. FIG. 13D shows that female BALB/c mice were immunized with MVA-EBV5-5 (5×10⁷ relative infectious units) on Day 0 and Day 28 (n=8), and blood was collected on Day 49. FIG. 13E shows that IgG binding levels to gp350, gB, and gp42 were measured using ELISA in individual mouse serum samples ( 1/900 dilution, bottom row), as compared to individual mouse serum samples from female BALB/c mice immunized with MVA and MVA-EBV5-2 presented in FIG. 2. Each dot represents the mean of duplicate measurements for each individual animal, with the median and interquartile range shown for each group. Statistical differences were determined using Kruskal-Wallis test (*=p<0.05, *** =p<0.001, ****=p<0.0001).

FIGS. 14A-14C show additional characterization of MVA-EBV5-2. FIG. 14A shows that the surface binding of the anti-gp350 mAb HB5, anti-gB mAb AMMO5, anti-gp42 mAb F-2-1, anti-gL mAb E1D1, and anti-gH mAb CL40 to BHK-21 cells infected with serially passaged MVA-EBV5-2 virus (p0, p4, p7, and p10) was measured using flow cytometry. Shown are representative quadrant plots from triplicate samples, with eGFP signal shown on the X-axis, a marker for MVA infection, and AF-647 signal shown on the Y-axis, corresponding to antibody staining. FIG. 14B shows the evaluation of MVA-EBV5-2 tropism, specifically the ability to infect two EBV susceptible cell lines (HEK-293 cells and Raji cells), as compared to MVA wildtype (MVA) and EBV, measured in the presence or absence of EBV-specific neutralizing antibodies 72A1 and AMMO1. Bar graphs represent the mean % infectivity+SD of triplicate measurements for each sample. FIG. 14C shows the evaluation of MVA-EBV5-2 storage stability. The MVA-EBV5-2 titer was measured in CEF cells after 1-or 5-month storage periods at −80° C. following virus production, via plaque-forming unit (PFU) immunostaining assay. Shown are representative micrographs of duplicate infected wells for each condition after immunostaining, together with the PFU count for each duplicate well per condition (well 1, W1; well 2, W2). Titers (PFU/ml) for both 1- and 5-month storage samples were calculated using the PFU well counts at the 10⁻⁸ dilution. Only positive well counts between 10-100 plaques were selected for measurement.

### DETAILED DESCRIPTION

The present technology includes recombinant Modified Vaccinia Ankara (rMVA) vectors configured to express EBV glycoproteins gp42, gL, gH, gp350, and gB. The present technology also includes vaccines comprising the rMVA vectors and methods of preventing or treating EBV infection by administering the vaccines and rMVA vectors of the present technology.

Upon first contact with the oral mucosa, EBV utilizes multiple glycoproteins to achieve entry into its two main target cells, epithelial and B cells. These glycoproteins, gp450, gB, gp42 and gHgL, are key targets of interest for developing an effective prophylactic vaccine⁷. In epithelial cells, the virus attaches to ephrin receptor A2 on target cells via the heterodimeric complex gHgL^{8,9}, which can also bind to non-muscle myosin heavy chain IIA (NMHC-IIA)¹⁰. Binding of gHgL to its target receptors then activates the fusogenic activity of gB⁷, bound to neuropilin 1 on the target cell¹¹, which culminates in viral entry⁷. In B cells, EBV attaches to completement receptor type 1 (CR1/CD35) and/or type 2 (CR2/CD21) via gp350^{12,13}, triggering endocytosis of the virion¹⁴. The fusion process is then carried out by gHgL in complex with gp42, which binds to MHC class II¹⁵⁻¹⁷, activating the fusogenic activity of gB. Of these five glycoproteins, gp350 dominated the field as the main immunogen tested during the first 20 years of EBV vaccine research⁶. This culminated in four Phase I/II clinical trials testing vaccines that targeted gp350 alone, but these were not successful in reducing EBV infection rates, failing to move to Phase III clinical trials and achieve licensure¹⁸⁻²¹. Knockout studies have shown that gp350 is not essential for viral entry²². However, these same studies have shown that gp350 does serve to enhance infection, and all five glycoproteins are targets of nAbs in both naturally-infected individuals and in animal antibody and vaccine studies^{6,23-35}. There are also reports that these glycoproteins can elicit cellular immune responses³⁶⁻⁴⁴. Based on this information, a robust immune response against multiple entry glycoproteins might be required for an EBV vaccine to achieve a sufficiently protective immune response against infection. Indeed, the field of EBV vaccine research has recently been shifting towards multivalent vaccine approaches^{6,45,46}, and the present technology includes multiple glycoproteins in a single vaccine to stimulate robust immune responses to prevent primary EBV infection and its associated diseases^{47,48}.

Previously, a multivalent virus-like particle (VLP) was developed that incorporated gp350, gB, gp42 and gHgL as a prophylactic EBV vaccine⁴⁸. The vaccine was immunogenic in immunized rabbits, eliciting glycoprotein-specific IgG with higher neutralizing activity in epithelial and B cells than IgGs elicited by a gp350-based vaccine, and on par with IgGs elicited by immunization with UV-inactivated EBV (UV-EBV). Despite these successes, the VLP production process was not optimal for large-scale manufacturing. In the present technology, the modified vaccinia Ankara (MVA) vector is used as a platform to express five target glycoproteins.

The present technology includes the design, development, characterization, and immunogenicity of an MVA-vectored multivalent vaccine candidate that incorporates gp350, gB, gp42 and gHgL. The vaccine is stable in ten viral passages, maintaining expression of all five glycoproteins in infected cells. In immunized BALB/c mice, the vaccine elicited glycoprotein-specific IgG against the target glycoproteins, with neutralizing activity in vitro that outperformed neutralizing activity elicited by immunization with UV-EBV. These results were replicated in rhesus-lymphocryptovirus (rhLCV)-negative rhesus macaque studies, where glycoprotein-specific IgG was detected in the saliva of immunized animals. The neutralizing activity elicited by the vaccine in rhesus macaques was further tested in vivo through passive immunization experiments in humanized mice; in two independent experiments testing different EBV challenge doses, the vaccine was able to protect humanized mice from EBV infection better than a gp350-based vaccine.

In some embodiments, the present technology includes a recombinant Modified Vaccinia Ankara (rMVA) vector comprising: (1) a first expression cassette comprising a single nucleic acid transcript encoding three EBV glycoproteins, wherein the EBV glycoproteins are gp42, gL, and gH, and wherein the nucleic acid transcript further comprises a self-cleaving 2A peptide between each of the EBV glycoproteins; and (2) a second expression cassette comprising a single nucleic acid transcript encoding two EBV glycoproteins, wherein the EBV glycoproteins are gp350 and gB, and wherein the nucleic acid transcript further comprises a self-cleaving 2A peptide between each of the EBV glycoproteins.

The recombinant rMVA vector may be derived from any MVA vector known in the art. A typical MVA strain which can be used according to the present technology for generating an rMVA is MVA 1974/NIH Clone 1 that has been deposited as ATCC Accession No.: PTA-5095 on Mar. 27, 2003 with the American Type Culture Collection (ATCC), 10801 University Blvd., Manassas, Va. 20110-2209, USA.

In some aspects, the rMVA includes one or more expression cassettes. The expression cassettes are configured to drive the expression of one or more antigens or proteins, for example EBV glycoproteins. In some aspects, the one or more expression cassettes drive expression of EBV glycoproteins selected from the group consisting of gp350, gB, gp42, and the gH/gL complex. In some aspects, the one or more expression cassettes drive expression of EBV glycoproteins selected from the group consisting of gp350, gB, gp42, gH, and gL. In some aspects, each expression construct comprises a single nucleotide transcript configured to drive the expression of the one or more EBV glycoproteins.

In some embodiments, the nucleic acid transcript further comprises a self-cleaving 2A peptide between each of the EBV glycoproteins. In some embodiments, 2A signal sequences that encode for the 2A peptide of food-and-mouth disease virus (F2A), equine rhinitis A virus (E2A), porcine teschovirus-1 (P2A), Thosea asigna virus (T2A), cytoplasmic polyhedrosis virus (BmCPV 2A), or flacherie virus (BmIFV 2A) can be used to link multiple genes under a single promoter. 2A signal sequences have been found in picornaviruses, insect viruses and type C rotaviruses. In some embodiments, a self-cleavage 2A peptide-derived sequence from Picornaviruses¹² is used to co-express EBV envelope glycoproteins including gp350, gB, gp42, gH and gL in native form. Bicistronic or multicistronic expression vectors can be used to express more than one gene product within a cell. In some embodiments, internal ribosome entry sites (IRES) can be introduced into one or more expression cassettes between nucleic acid sequences encoding EBV envelope glycoproteins that are co-expressed, flanking the sequences encoding the glycoproteins. Although IRES can be used to link the expression of multiple genes under a single promoter, the use of multiple IRES sequences might be limited by size constraints, instability due to its relatively larger size comparing to 2A signal sequences, and/or difference in expression levels between the genes located before and after an IRES.

Additionally, a furin cleavage site preceding the 2A signal sequences can be incorporated to remove the 2A peptides following self-processing of the 2A-linked polyproteins. Furin is an enzyme that occurs in the Golgi apparatus and cleaves at very short signal peptides such as KKKR or RKKR motif. Furin cleavage contributes to protein processing and maturation. These short signal peptides can be added to the N-terminus of the 18-22 amino acid long 2A skipping signals so that they are removed following 2A-mediated processing of the EBV envelope glycoproteins, except for one or two remaining amino acids. The resultant product can be even more “native.” Although it is preferred that the 2A-linked glycoproteins are expressed all from one vector through the use of one or more expression cassettes, it is also possible to express the 2A-linked subunits from two or more separate vectors.

By exploiting the ribosomal skipping mechanism conferred by 2A peptides, an approach of co-expressing the EBV envelope glycoproteins as only one or two self-processing polyproteins is disclosed herein. The 2A ribosomal skipping system is widely-used to express multi-protein complexes due to the relative small sizes of 2A peptides (18-22 amino acids) and because it allows stoichiometric expression of the individual 2A-linked subunits. In some embodiments, P2A-linked DNA sequences of two or more EBV envelope glycoproteins are co-expressed and efficiently cleaved and transported to the cell surface. In some embodiments, the DNA sequences encoding the EBV envelope glycoproteins are codon-optimized. In some embodiments, the co-expressed EBV envelope glycoproteins are self-assembled into surface complexes, including gp42-gH/gL and gB-gH/gL.

In some aspects, the rMVA includes a first expression cassette and a second expression cassette that are each configured to drive the expression of one or more antigens or proteins, for example EBV glycoproteins. In some aspects, the first expression cassette and the second expression cassette in combination drive the expression of EBV glycoproteins gp350, gB, gp42, and the gH/gL complex. In some aspects, the first expression cassette and the second expression cassette in combination drive the expression of EBV glycoproteins gp350, gB, gp42, gH, and gL. In some aspects, the first expression cassette is configured to drive the expression of EBV glycoproteins selected from the group of gp42, gL, and gH.

In some aspects, the first expression construct comprising a single nucleic acid transcript encoding three EBV glycoproteins selected from the group consisting of gp350, gB, gp42, gH, and gL. In some aspects, the first expression construct comprises a single nucleic acid transcript encoding gp42, gL, and gH. In some aspects, the first expression constructs further comprises a self-cleaving 2A peptide between each of the three EBV glycoproteins. For example, the first expression construct may comprise a single nucleic acid transcript having two self-cleaving 2A peptides between the three EBV glycoproteins. In some aspects, the first expression construct comprises a single nucleic acid transcript sequentially encoding gH, gL, and gp42. As used herein, “sequentially encoding” means that the EBV glycoproteins are expressed sequentially in the recited order from the promoter with or without 2A peptides between. For example, a transcript sequentially encoding gH, gL, and gp42 encodes gH first, gL second, and gp42 third.

In some aspects, the second expression construct comprising a single nucleic acid transcript encoding two EBV glycoproteins selected from the group consisting of gp350, gB, gp42, gH, and gL. In some aspects, the second expression construct comprises a single nucleic acid transcript encoding gp350 and gB. In some aspects, the second expression constructs further comprises a self-cleaving 2A peptide between the two EBV glycoproteins. For example, the second expression construct may comprise a single nucleic acid transcript having one self-cleaving 2A peptide between the two EBV glycoproteins. In some aspects, the second expression construct comprises a single nucleic acid transcript sequentially encoding gp350 and gB.

In some aspects, the one or more expression cassettes are inserted into one or more MVA insertion sites. In some aspects, the one or more expression cassettes are inserted into one or more MVA intergenic regions (IGRs). In some aspects, the one or more expression cassettes are inserted into an MVA IGRs selected from the group consisting of the 69R/70L (G1L) genomic site and the 64L/65L (IGR3) MVA genomic site. In some aspects, the first expression cassette is inserted into the IGR3 insertion site. In some aspects, the second expression cassette is inserted into the G1L insertion site.

In some embodiments, the one or more expression cassettes comprise a promoter. Various suitable eukaryotic cell promoters can be used, including but not limited to, immediate-early I promoter of human CMV or the chicken beta actin promoter, promoters of vaccinia virus (mH5, pSyn, P11, p7.5), etc. In some aspects, the one or more expression cassettes comprise an mH5 promoter.

In some embodiments, the rMVA vector is genetically and translationally stable. As used herein, the rMVA vector is “genetically and translationally stable” if it is capable of undergoing multiple rounds of viral passage without experiencing substantial degradation of the structure of the rMVA or loss of its ability to express the EBV glycoproteins. In some embodiments, the rMVA vector is genetically and translationally stable in 5, 6, 7, 8, 9, 10, or more than 10 viral passages. In some embodiments, the rMVA vector is genetically and translationally stable in 10 viral passages. and expresses each of the EBV glycoproteins. In some embodiments, the rMVA continues to express all the EBV glycoproteins after 10 viral passages.

Expression systems, vectors, vaccines for use in preventing or treating EBV infections are provided herein. In some embodiments, the present technology includes compositions comprising the rMVA vectors of the present technology. In some embodiments, the present technology includes vaccine or immunogenic fragments comprising the rMVA vectors of the present technology.

In some embodiments, the vectors and vaccines of the present technology can stimulate both humoral (antibody) and T cell-mediated immunity, and generate both prophylactic and therapeutic antiviral responses against EBV infection and EBV-associated malignancies. In some embodiments, the immunization regimens of the present technology elicit an IgG response against the EBV glycoproteins expressed by the rMVA vector. In some embodiments, the immunization regimens of the present technology elicit an IgG response of a IgG1 subtype against the EBV glycoproteins expressed by the rMVA vector. In some embodiments, the immunization regimens of the present technology elicit an IgG response of a IgG2a subtype against the EBV glycoproteins expressed by the rMVA vector. In some embodiments, the immunization regimens of the present technology elicit a Th1- and Th2-type immune response.

According to the embodiments described herein, an immunization regimen is provided. The immunization regimen includes rMVA vector configured to express EBV glycoproteins gp42, gL, gH, gp350, and gB. The immunization regimen may be administered via prime/boost homologous (e.g. using only the same vaccine type) or heterologous (e.g. using different vaccine types) vaccination. The immunization regimen may be administered in a dose vaccination schedule involving two or more immunizations, which may be administered 2 weeks to 6 months apart. Other suitable immunization schedules or regimens that are known in the art may be used according to the embodiments described herein by those skilled in the art.

The vaccine composition as described herein may comprise a therapeutically effective amount of a rMVA vector as described herein, and may further comprise a pharmaceutically acceptable carrier according to a standard method. Examples of acceptable carriers include physiologically acceptable solutions, such as sterile saline and sterile buffered saline.

In some embodiments, the vaccine or pharmaceutical composition may be used in combination with a pharmaceutically effective amount of an adjuvant to enhance the anti-EBV effects. Any immunologic adjuvant that may stimulate the immune system and increase the response to a vaccine, without having any specific antigenic effect itself may be used as the adjuvant. Many immunologic adjuvants mimic evolutionarily conserved molecules known as pathogen-associated molecular patterns (PAMPs) and are recognized by a set of immune receptors known as Toll-like Receptors (TLRs). Examples of adjuvants that may be used in accordance with the embodiments described herein include Alum, Freund's complete adjuvant, Freund's incomplete adjuvant, double stranded RNA (a TLR3 ligand), LPS, LPS analogs such as monophosphoryl lipid A (MPL) (a TLR4 ligand), flagellin (a TLR5 ligand), lipoproteins, lipopeptides, single stranded RNA, single stranded DNA, imidazoquinolin analogs (TLR7 and TLR8 ligands), CpG DNA (a TLR9 ligand), Ribi's adjuvant (monophosphoryl-lipid A/trehalose dicorynoycolate), glycolipids (α-GalCer analogs), unmethylated CpG islands, oil emulsion, liposomes, virosomes, saponins (active fractions of saponin such as QS21), muramyl dipeptide, alum, aluminum hydroxide, squalene, BCG, cytokines such as GM-CSF and IL-12, chemokines such as MIP 1-α and RANTES, activating cell surface ligands such as CD40L, N-acetylmuramine-L-alanyl-D-isoglutamine (MDP), and thymosinα. The amount of adjuvant used can be suitably selected according to the degree of symptoms, such as softening of the skin, pain, erythema, fever, headache, and muscular pain, which might be expressed as part of the immune response in humans or animals after the administration of this type of vaccine.

In further embodiments, use of various other adjuvants, drugs or additives with the vaccine of the invention, as discussed above, may enhance the therapeutic effect achieved by the administration of the vaccine or pharmaceutical composition. The pharmaceutically acceptable carrier may contain a trace amount of additives, such as substances that enhance the isotonicity and chemical stability. Such additives should be non-toxic to a human or other mammalian subject in the dosage and concentration used, and examples thereof include buffers such as phosphoric acid, citric acid, succinic acid, acetic acid, and other organic acids, and salts thereof; antioxidants such as ascorbic acid; low molecular weight (e.g., less than about 10 residues) polypeptides (e.g., polyarginine and tripeptide) proteins (e.g., serum albumin, gelatin, and immunoglobulin); amino acids (e.g., glycine, glutamic acid, aspartic acid, and arginine); monosaccharides, disaccharides, and other carbohydrates (e.g., cellulose and derivatives thereof, glucose, mannose, and dextrin), chelating agents (e.g., EDTA); sugar alcohols (e.g., mannitol and sorbitol); counterions (e.g., sodium); nonionic surfactants (e.g., polysorbate and poloxamer); antibiotics; and PEG.

The vaccine or pharmaceutical composition containing a rMVA vector described herein may be stored as an aqueous solution or a lyophilized product in a unit or multiple dose container such as a sealed ampoule or a vial.

The expression systems, vectors and vaccines described herein may be used to treat or prevent any EBV infection or conditions associated with EBV infection such as EBV+lymphomas, carcinomas, PTLDs, multiple sclerosis among other diseases.

As used herein, the term “subject” is an animal. In some embodiments, the subject is a mammal. In some embodiments, the subject is human.

The term “an effective amount” as used herein refers to an amount of a composition that produces a desired effect. For example, a population of cells may be infected with an effective amount of a viral vector to study its effect in vitro (e.g., cell culture) or to produce a desired therapeutic effect ex vivo or in vitro. An effective amount of a composition may be used to produce a prophylactic or therapeutic effect in a subject, such as preventing or treating a target condition, alleviating symptoms associated with the condition, or producing a desired physiological effect. In such a case, the effective amount of a composition is a “therapeutically effective amount,” “therapeutically effective concentration” or “therapeutically effective dose.” The precise effective amount or therapeutically effective amount is an amount of the composition that will yield the most effective results in terms of efficacy of treatment in a given subject or population of cells. This amount will vary depending upon a variety of factors, including but not limited to the characteristics of the composition (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further an effective or therapeutically effective amount may vary depending on whether the composition is administered alone or in combination with another composition, drug, therapy or other therapeutic method or modality. One skilled in the clinical and pharmacological arts will be able to determine an effective amount or therapeutically effective amount through routine experimentation, namely by monitoring a cell's or subject's response to administration of a composition and adjusting the dosage accordingly. For additional guidance, see Remington: The Science and Practice of Pharmacy, 21^{st} Edition, Univ. of Sciences in Philadelphia (USIP), Lippincott Williams & Wilkins, Philadelphia, PA, 2005, which is hereby incorporated by reference as if fully set forth herein.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. Treatment may also mean a prophylactic or preventative treatment of a condition.

In some embodiments, the vaccine or pharmaceutical composition described herein may be used in combination with other known pharmaceutical products, such as immune response-promoting peptides and antibacterial agents (synthetic antibacterial agents). The vaccine or pharmaceutical composition may further comprise other drugs and additives. Examples of drugs or additives that may be used in conjunction with a vaccine or pharmaceutical composition described herein include drugs that aid intracellular uptake of the composition or vaccine disclosed herein, liposome and other drugs and/or additives that facilitate transfection, (e.g., fluorocarbon emulsifiers, cochleates, tubules, golden particles, biodegradable microspheres, and cationic polymers).

In some embodiments, the vaccine composition or pharmaceutical composition described herein may be administered by directly injecting a rMVA vector suspension prepared by suspending the rMVA in PBS (phosphate buffered saline) or saline into a local site, by nasal or respiratory inhalation, or by intravascular (i.v.) (e.g., intra-arterial, intravenous, and portal venous), subcutaneous (s.c.), intracutaneous (i.c.), intradermal (i.d.), intraperitoneal (i.p.) or intramuscular (i.m.) administration. The vaccine or pharmaceutical composition of the present invention may be administered more than once. More specifically, after the initial administration, one or more additional vaccinations may be given as a booster. One or more booster administrations can enhance the desired effect. After the administration of the vaccine or pharmaceutical composition, booster immunization with a pharmaceutical composition containing the rMVA vector as described herein may be performed.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1

### Generation and Characterization of Multivalent MVA-Based Candidate, MVA-EBV5-2

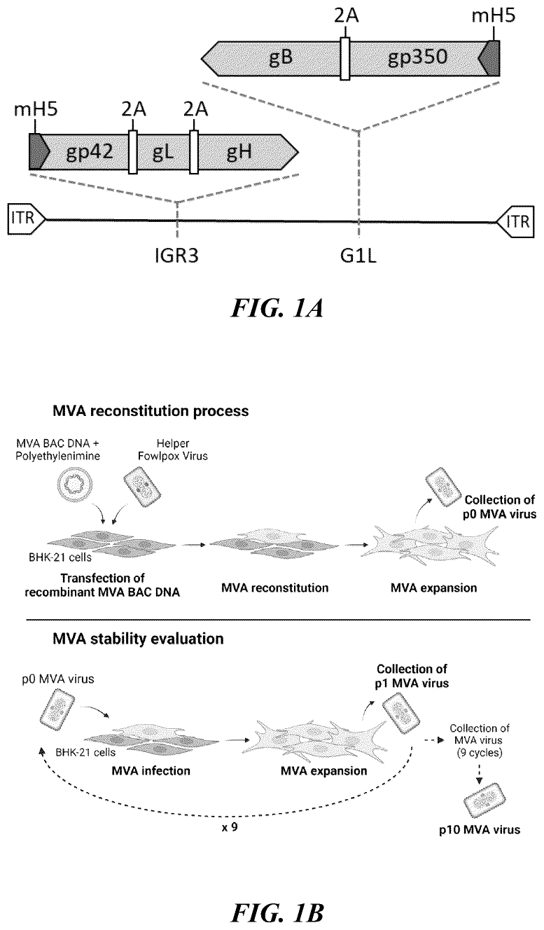

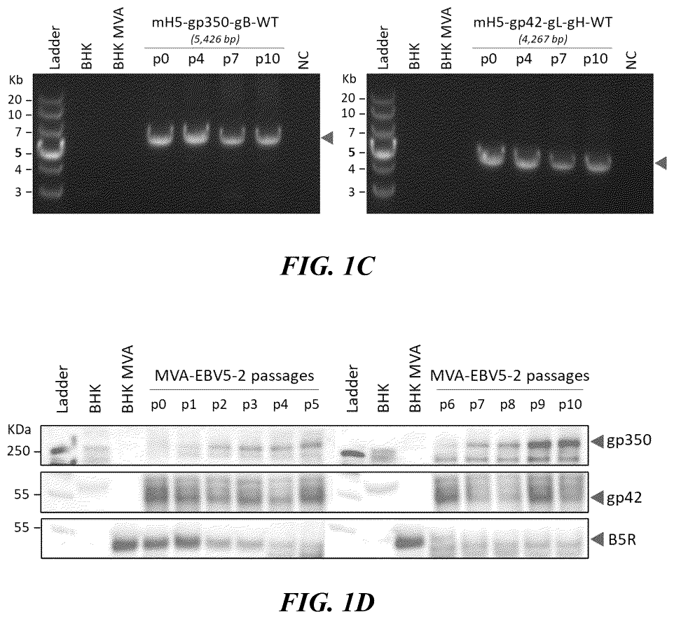

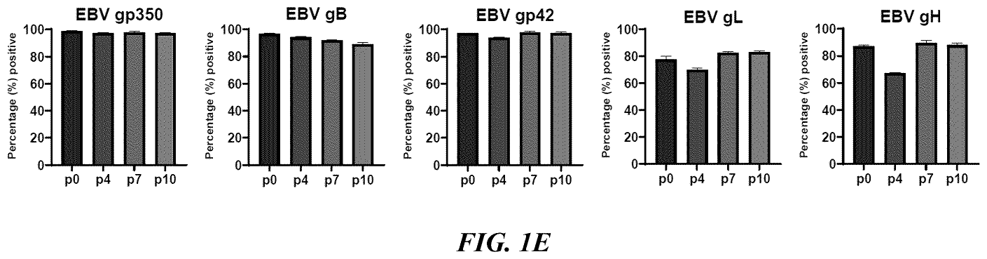

Using eGFP-positive MVA-BAC-TK⁶¹, a recombinant MVA vector was generated to incorporate five EBV glycoproteins involved in viral entry of B cells and epithelial cells, gp350, gB, and gp42gHgL (MVA-EBV5-2). Two polycistronic glycoprotein expression cassettes that code for either gp42, gL and gH (trivalent) or gp350 and gB (bivalent) in wildtype form (FIG. 1A) were cloned into the MVA BAC via en passant mutagenesis, a homologous recombination-based cloning system⁶². Each cassette codes for each corresponding set of glycoproteins in a single transcript under the control of an mH5 promoter, a promoter that has been shown to stabilize transgene in recombinant MVA constructs⁶³. To allow for individual glycoprotein release upon transcription, the glycoprotein sequences are interspersed by unique self-cleaving 2A peptides⁶⁴.

To verify sequence fidelity before viral reconstitution, Sanger sequencing of each expression cassette in the final MVA-EBV5-2 construct was performed. Resulting sequence analysis revealed full alignment to the expected sequence (data not shown), except for the presence of a Δ210bp truncation (Δa.a.513-549) in the gp350ectodomain. Given the presence of several 29-bp repetitive sequences in gp350, use of the en passant system can result in homologous recombination of these repetitive sequences, allowing the emergence of several truncated gp350 products. However, these truncations are found within the gp220 splice site, which has been excluded from other recent gp350-targeting vaccines^{45,65,66}, and do not overlap with any of the previously identified gp350 neutralizing epitopes³⁰; thus it is unlikely that they affect antigen immunogenicity. Therefore, the recombinant MVA-EBV5-2 DNA was transfected into BHK-21 cells, MVA-permissive cells, and the virus was reconstituted with helper fowlpox virus, generating passage 0 (p0) virus (FIG. 1B).

Following reconstitution of p0 virus, it was determined whether the inserted glycoprotein expression cassettes were stable in MVA-EBV5-2 after several viral passages, an important quality for large-scale manufacturing of viral vector vaccines. MVA-EBV5-2 was serially passaged ten times in BHK-21 cells beginning with p0 (FIG. 1B), a commonly tested number of passages for MVA^{63,67}, and infected cells were collected at each passage to isolate DNA and total protein for genetic and translational stability assessment. To first assess genetic stability, the isolated DNA was used as a template to PCR-amplify each expression cassette and determine the size of the amplicons at each passage. PCR amplification of the expression cassettes in p0, p4, p7, and p10 DNA resulted in amplicons of the expected size for both trivalent and bivalent cassettes, at all passages tested (FIG. 1C). This suggested that sequence fidelity was maintained in up to 10 viral passages. These results were further confirmed by full genome sequencing of p0 and p10 DNA, which revealed complete alignment of the DNA to the expected sequence (data not shown). Next, translational stability of the viruses was assessed by performing immunoblot and flow cytometry analysis of infected BHK-21 cells. Expression of gp350 and gp42 was confirmed in cells infected with p1-p10 of MVA-EBV5-2 by immunoblot (FIG. 1D). Moreover, flow cytometry analysis using antibodies against all five target glycoproteins confirmed expression of all five glycoproteins in cells infected with p0-p10 (FIG. 1E). Taken together, these results confirm MVA-EBV5-2 to be fully stable, genetically and translationally, through ten viral passages.

### Example 2

### Immunogenicity of MVA-EBV5-2 in BALB/c Mice

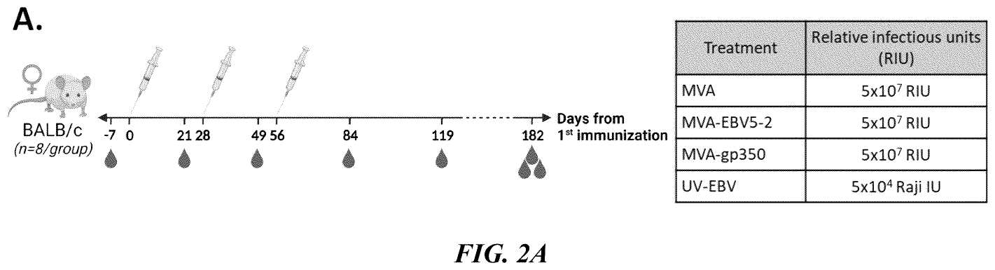

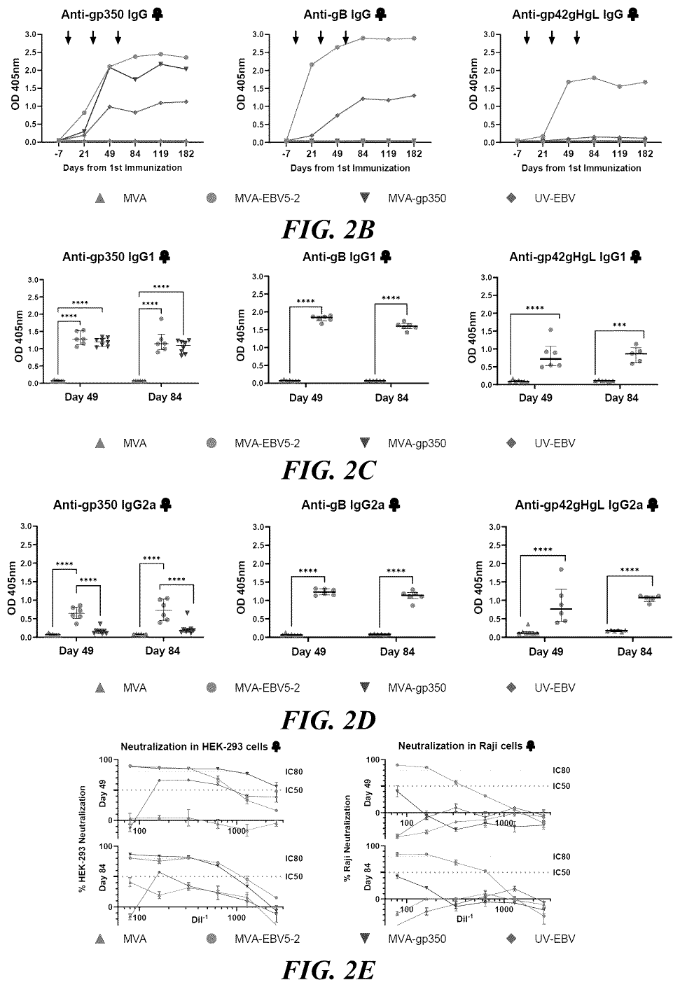

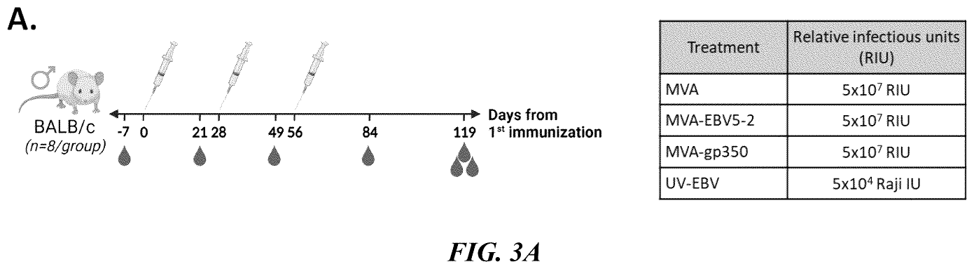

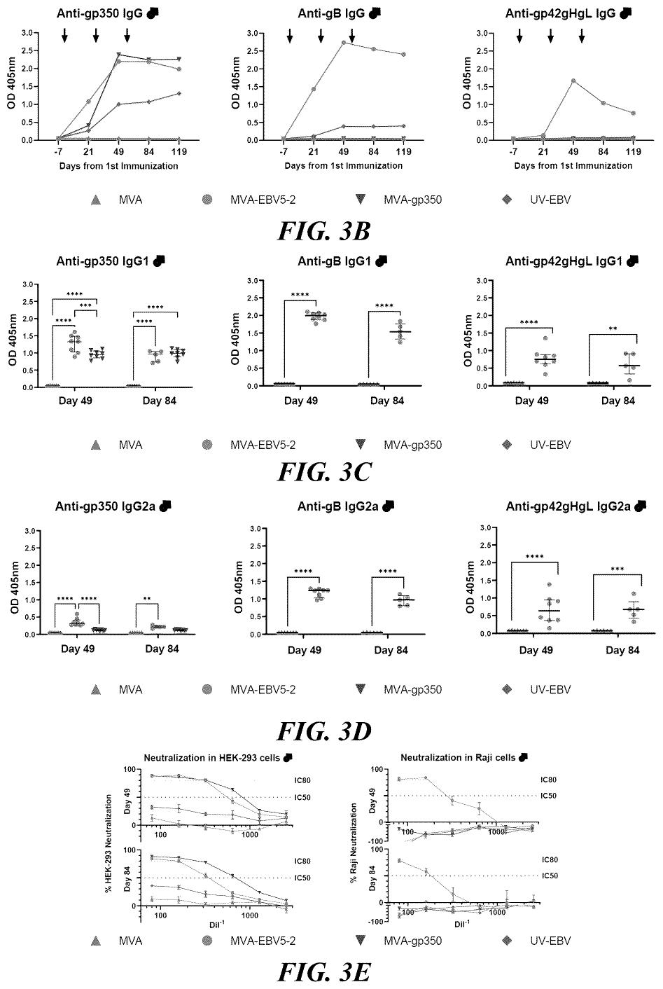

To begin assessing the immunogenicity of MVA-EBV5-2, both female (FIG. 2A) and male (FIG. 3A) BALB/c mice were immunized with the vaccine three times in 4-week intervals. Alternatively, mice were immunized with an MVA vector that expresses gp350 (Δ210 truncation, MVA-gp350) as a positive control representative of previous clinical vaccine candidates, UV-inactivated EBV (UV-EBV) as a positive control that contains all five targeted glycoproteins, or empty MVA virus as a negative control.

To assess the levels of glycoprotein-specific IgG elicited by the different treatments, ELISA was performed using the sera of immunized mice. First, the kinetics of the humoral immune response were analyzed throughout the observation period using pooled serum of female and male mice (FIG. 2B and 3B). As shown, MVA-EBV5-2 was successful in generating IgG against gp350, gB and the gp42gHgL complex at similar levels. The levels of IgG tended to increase after the first and second dose, with no increment after the third. The same kinetics were observed with MVA-gp350, which elicited IgG against gp350 but not against the other glycoproteins. UV-EBV only induced the production of IgG against gp350 and gB, while no specific IgG were detected on the MVA group at any timepoint.

To further characterize the humoral immune response before and after third immunization and determine the EBV glycoprotein-specific IgG isotypes elicited by the MVA vaccines, the individual mouse serum levels of IgG1 (FIG. 2C and 3C) and IgG2a (FIG. 2D and 3D) were evaluated by ELISA at Days 49 and 84. MVA-EBV5-2-immunized mice exhibited both IgG1 and IgG2a antibodies against all glycoproteins in both sexes; in contrast, MVA-gp350-immunized mice mainly exhibited glycoprotein-specific IgG1. IgG levels did not increase after the third MVA-EBV5-2 dose (Day 84) in either sex, which together with the kinetic analyses, suggested that a third MVA-EBV5-2 immunization did not further boost IgG responses; thus, the number of MVA-EBV5-2 doses were reduced to two in the next immunogenicity experiment, which is representative of regimens used in the clinic for other MVA-based vaccines^{56,60,68}. The ability of pooled sera to neutralize EBV infection in HEK-293 and Raji cells was also measured

Finally, to determine whether the elicited antibodies exhibited EBV neutralizing activity, in vitro neutralization assays were performed in HEK-293 epithelial cells and Raji B cells against Akata-EBV-eGFP virus using serially diluted pooled mouse sera from samples collected on Days 49 and 84 in both female and male mice (FIG. 2E and 3E). As reported in a systematic review, most EBV vaccine studies do not provide viral infectivity of the EBV batch used in neutralization assays⁶; to ensure rigor, experimental transparency and interpretability of results, it is important that vaccine studies report the full spectrum of experimental details for neutralization assays. Here, serum-free Akata-EBV-eGFP infectivity was measured to be 13.2% and 17.6% in HEK-293 cells, and 28% and 16.8% in Raji cells, for female and male mice assays respectively. Neutralization was calculated using these values as maximum infection rate (100%). As shown, sera from female mice immunized with MVA-EBV5-2 neutralized EBV infection in both HEK-293 and Raji cells in a dose-dependent manner, better than sera from mice immunized with UV-EBV. Indeed, UV-EBV neutralizing activity was lower overall in HEK-293 cells, and virtually non-existent in Raji cells. MVA-gp350 performed similarly to MVA-EBV5-2 in HEK-293 cells, but displayed much lower neutralizing activity in Raji cells. Similar results were observed in male mice, although neutralizing activity was lower in all male groups overall, and the MVA-gp350 group did not display any neutralizing activity in Raji cells. Next, the immunogenicity of MVA-EBV5-2 was determined in a NHP model.

### Example 3

Immunogenicity of MVA-EBV5-2 in rhLCV-Negative Rhesus Macaques

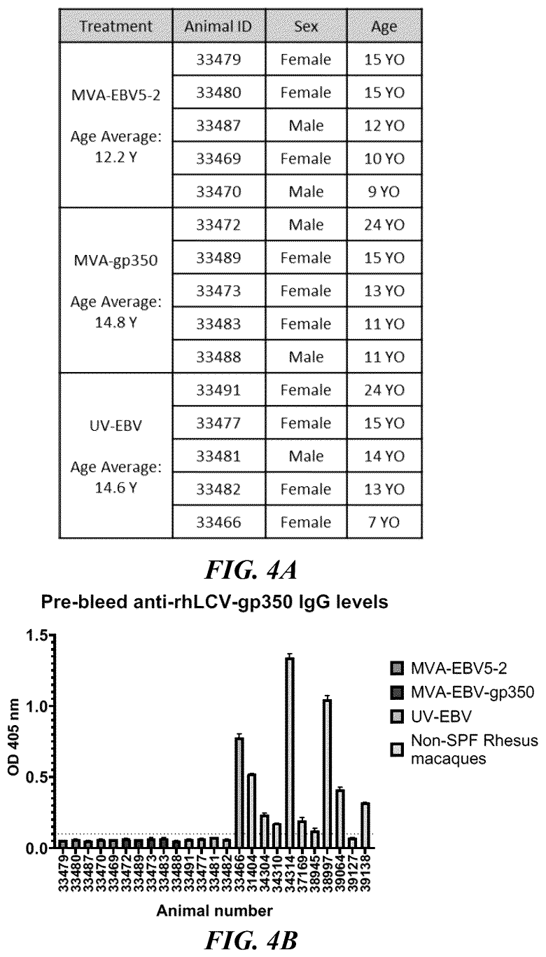

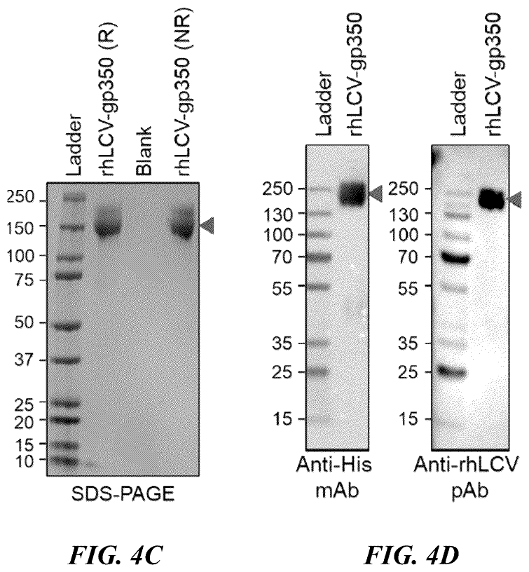

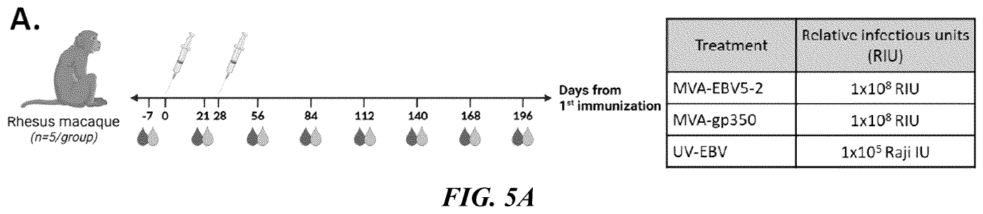

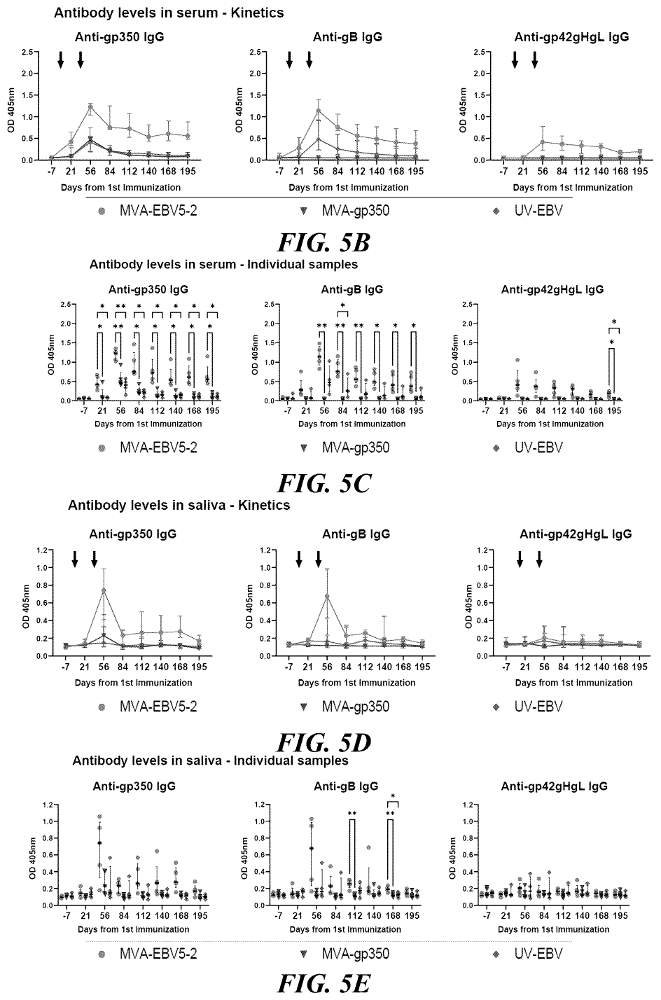

To further validate MVA-EBV5-2 as a vaccine candidate with clinical potential, an immunogenicity study was performed in rhesus-lymphocryptovirus (rhLCV)-negative rhesus macaques. Until recently^{29,45,65,66}, most previous pre-clinical EBV vaccine studies employing NHPs did not address the fact that NHPs are hosts to pervasive EBV-homologue LCVs that can result in antigenic cross-reactivity⁶⁹, and thus affect vaccine immunogenicity assessments. To avoid this issue, the rhesus macaque cohort was pre-screened by the Oregon National Primate Research Center for rhLCV, the rhesus-specific EBV homologue, and maintained in expanded specific-pathogen-free conditions throughout the study to avoid rhLCV transmission. Fifteen animals were originally enrolled in the study and distributed into three treatment groups: UV-EBV, MVA-gp350, and MVA-EBV5-2 (FIG. 4A). Macaques were immunized twice in a 4-week interval, and both blood and saliva was collected for humoral immune response assessment throughout the observation period (FIG. 5A). To confirm rhLCV sero-negativity at the beginning of the study, an rhLCV-specific ELISA was performed using Day −7 serum from each animal (FIG. 4B); although most rhesus macaques showed no rhLCV seroreactivity, one animal from the UV-EBV-immunized group (ID 33466) displayed significant levels of anti-rhLCV-gp350 IgG on par with levels in macaques from an independent non-SPF colony, and thus it was excluded from subsequent immunogenicity analyses. The protein used for rhLCV screening by ELISA has high purity and it is recognized by an anti-rhLCV polyclonal serum confirming its identity (FIG. 4C and 4D).

To assess the levels of IgG against gp350, gB, and gp42gHgL complex elicited by the vaccine, ELISA was performed using the serum and saliva of immunized macaques. In the serum (FIG. 5B and 5C), MVA-EBV5-2 induced an increase of anti-gp350,-gB and -gp42gHgL IgG in all animals, reaching maximum levels after the second dose. High levels of glycoprotein-specific IgG were found even 6 months after second dose. As was observed in BALB/c mice, MVA-gp350 elicited IgG against gp350 but not against the other glycoproteins, while UV-EBV induced an IgG response against gp350 and gB alone. In the saliva, MVA-EBV5-2 elicited an increase of anti-gp350 and -gB IgG levels after the second dose in 4/5 animals vs. ¼ animals on UV-EBV group (FIG. 5C and 5D).

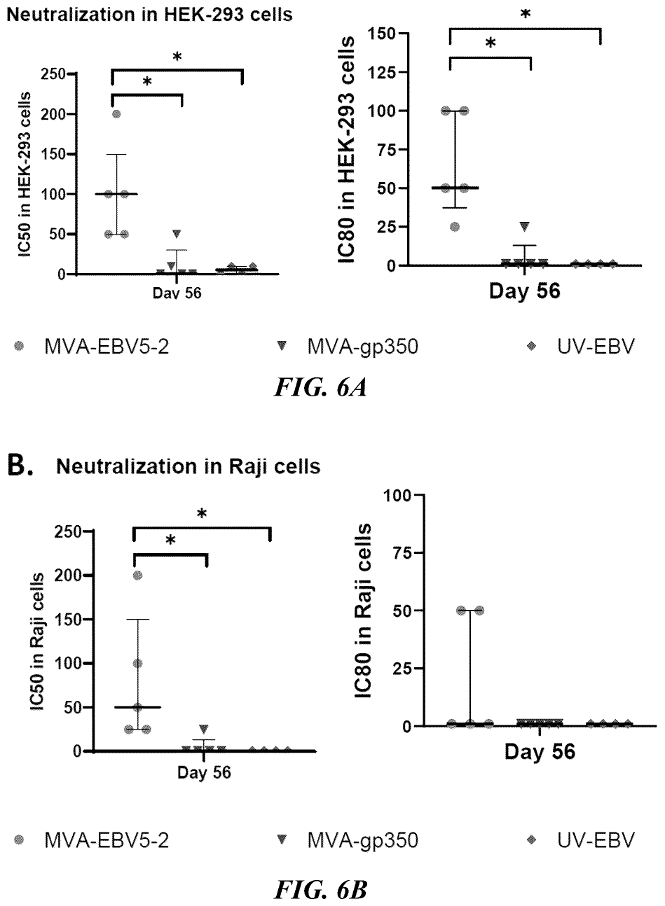

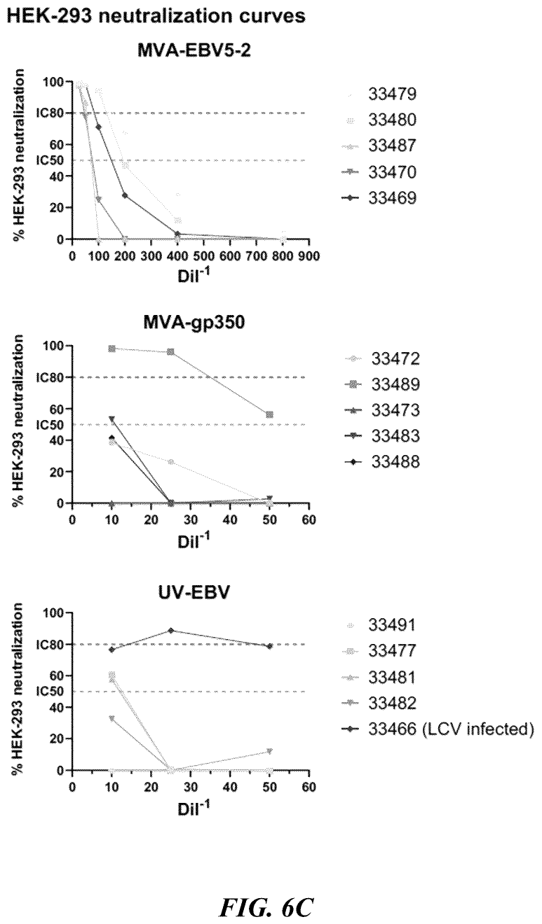

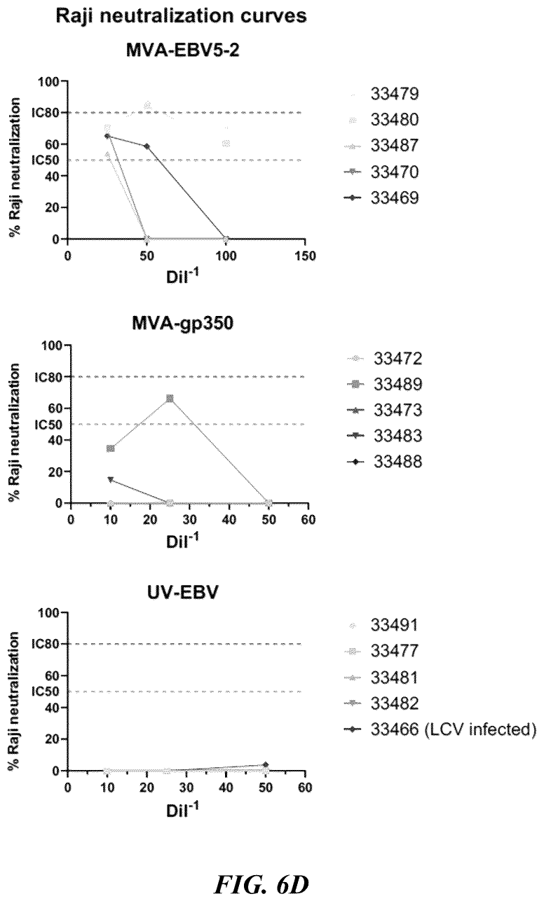

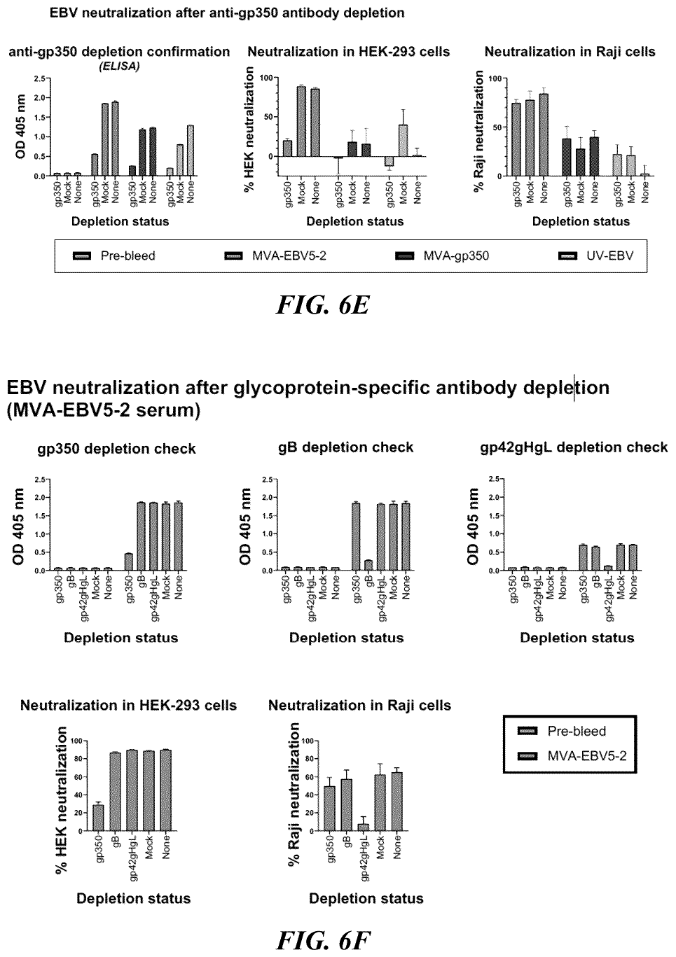

To determine if serum antibodies elicited by MVA-EBV5-2 in rhesus macaques had neutralizing potential, in vitro neutralization assays were performed in HEK-293 (FIG. 6A and 6C) and Raji cells (FIG. 6B and 6D) using serially diluted individual serum samples collected on Day 56. The serum-free EBV-Akata infectivity in these experiments were 25.0% and 5.3% for HEK-293 and Raji cells, respectively. To eliminate the sera effect, the IC50 and IC80 results are based on the neutralization achieved using the serum collected on Day −7 (Pre-immune) versus Day 56 serum from the same animal at the same dilution. In both HEK-293 and Raji assays, serum samples from MVA-EBV5-2 animals outperformed samples from MVA-gp350 and UV-EBV animals in neutralizing EBV infection, confirming that MVA-EBV5-2 elicited higher levels of neutralizing antibodies than MVA-gp350 and UV-EBV.

Given that gp350 is the main target of neutralizing antibodies against EBV B cell infection in EBV-seropositive individuals, the contribution of gp350-specific antibodies to the neutralizing activity of immune rhesus macaque sera from the different treatment groups was determined. To achieve this, Day 56 immune sera was pooled for each rhesus macaque group and incubated each serum pool with nitrocellulose membranes either coated with gp350 protein and blocked with bovine serum album (BSA) (gp350 depletion), or only blocked with BSA (mock depletion). After verifying that gp350-specific antibodies were depleted from the sera pools (FIG. 6E, left panel), neutralization assays were repeated using depleted sera, mock-depleted sera, and undepleted sera in both HEK-293 and Raji cells (FIG. 6E, middle and right panel), measuring serum-free Akata-EBV-eGFP infectivity as 16.5% and 4.6% for HEK-293 and Raji cells, respectively. In MVA-EBV5-2 sera, most of the neutralizing activity was lost in HEK-293 cells after anti-gp350 antibody depletion; in Raji cells however, there was almost no reduction in neutralizing activity when comparing depleted versus undepleted sera. MVA-gp350 sera lost all neutralizing activity in HEK-293 cells after depletion, but neutralizing activity in Raji cells was not affected.

Considering that anti-gp350 antibody depletion of MVA-EBV5-2 sera did not significantly reduce neutralization in Raji cells, the contribution of the different target glycoproteins by performing additional depletion experiments was determined (FIG. 6F). After confirming the depletion of anti-gp350,-gB and -gp42gHgL antibodies in MVA-EBV5-2 sera by ELISA (FIG. 6F, top panel), the neutralization assays were repeated in both HEK-293 and Raji cells (FIG. 6F, bottom panel), reaching a serum-free Akata-EBV-eGFP infectivity of 11.7% and 30.4% in each cell line respectively. Results confirmed that most of the neutralizing activity in HEK-293 cells is due to the presence of anti-gp350 antibodies, while in Raji cells this effect is mainly associated with anti-gp42gHgL antibodies.

### Example 4

Passive Transfer of MVA-EBV5-2-Immune Rhesus Macaque Sera Protects Humanized Mice Against EBV Infection Better than Immune Sera from MVA-gp250-Immunized Rhesus Macaques

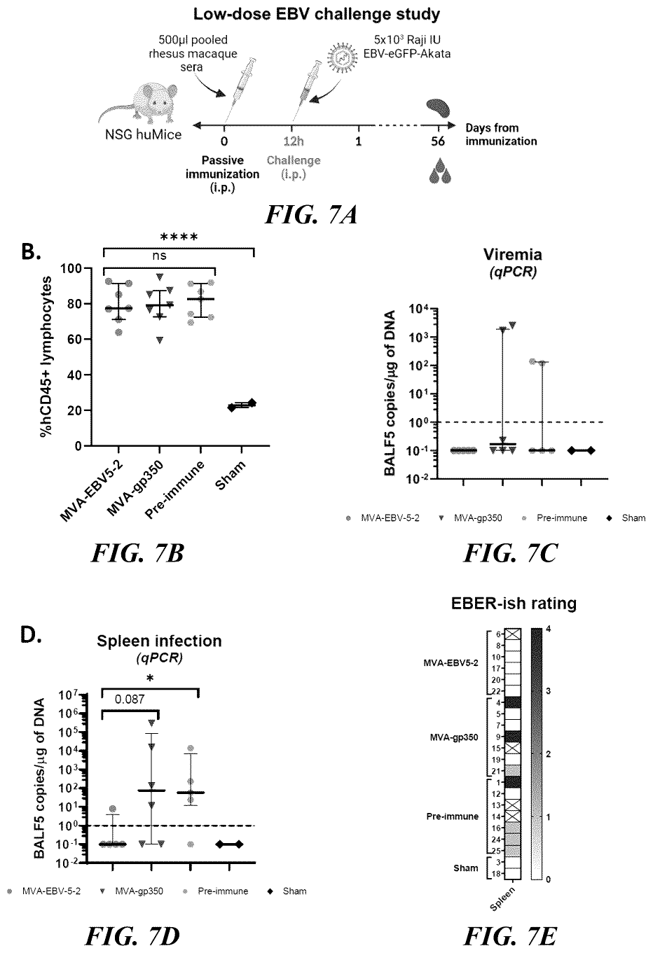

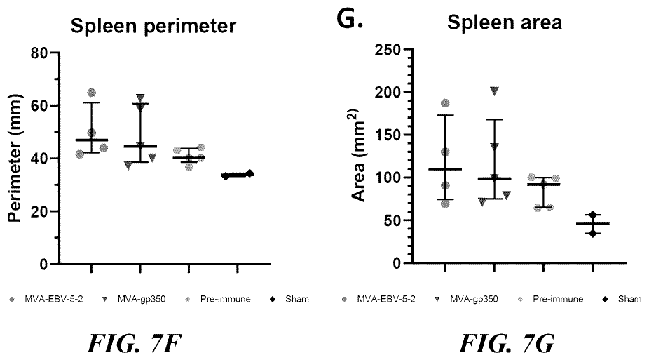

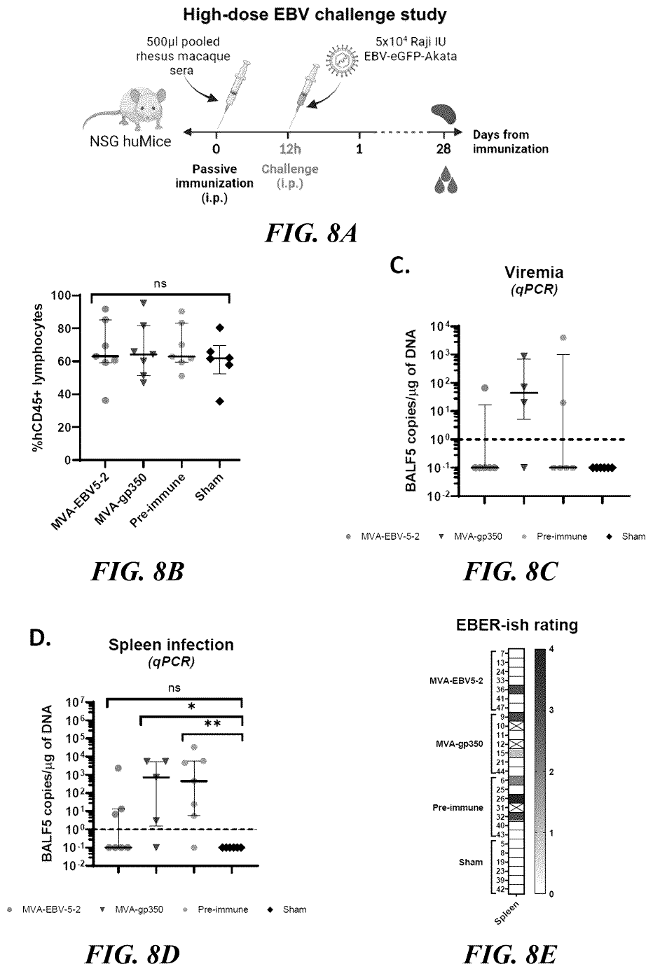

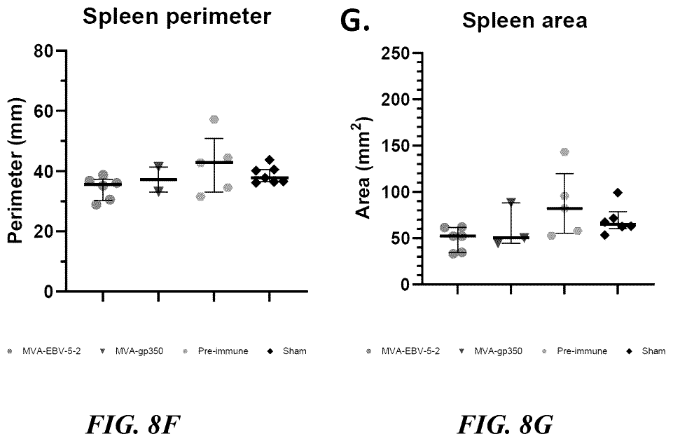

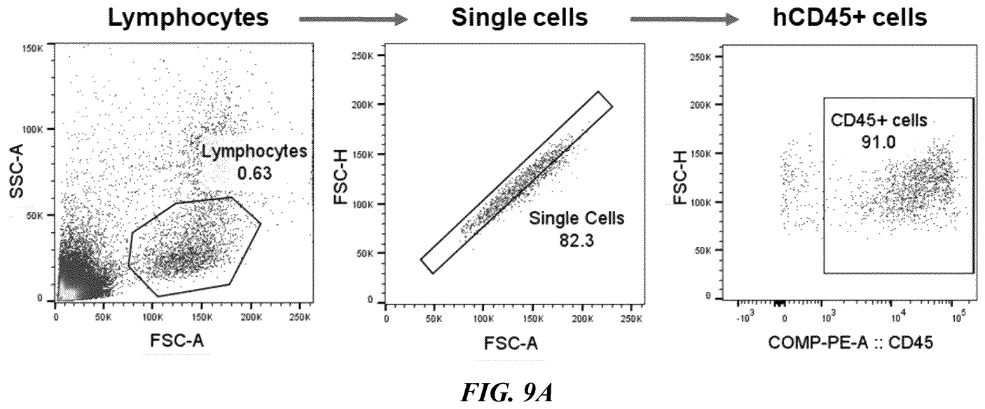

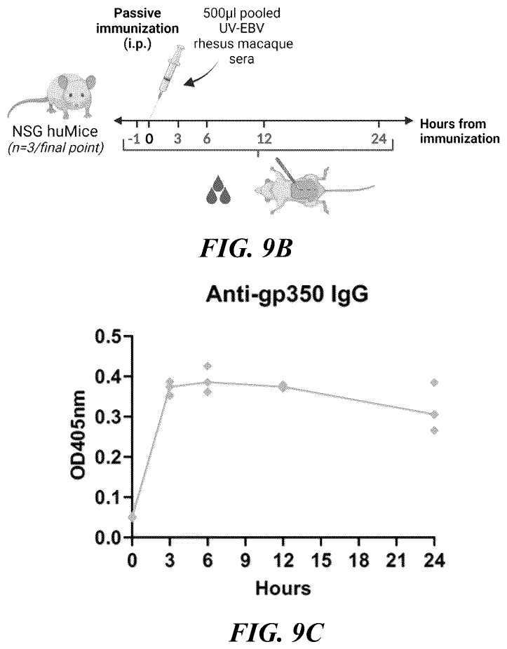

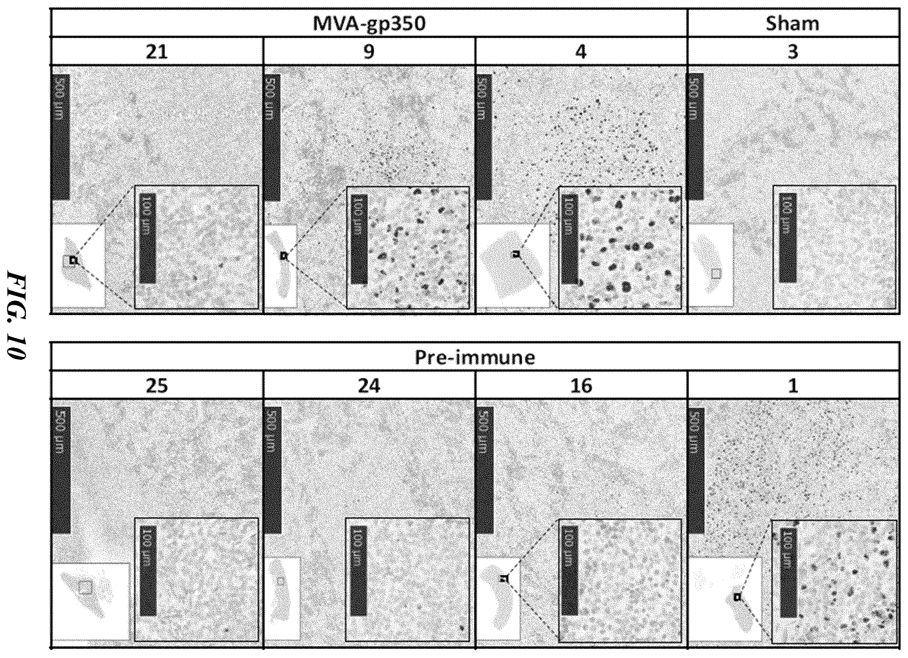

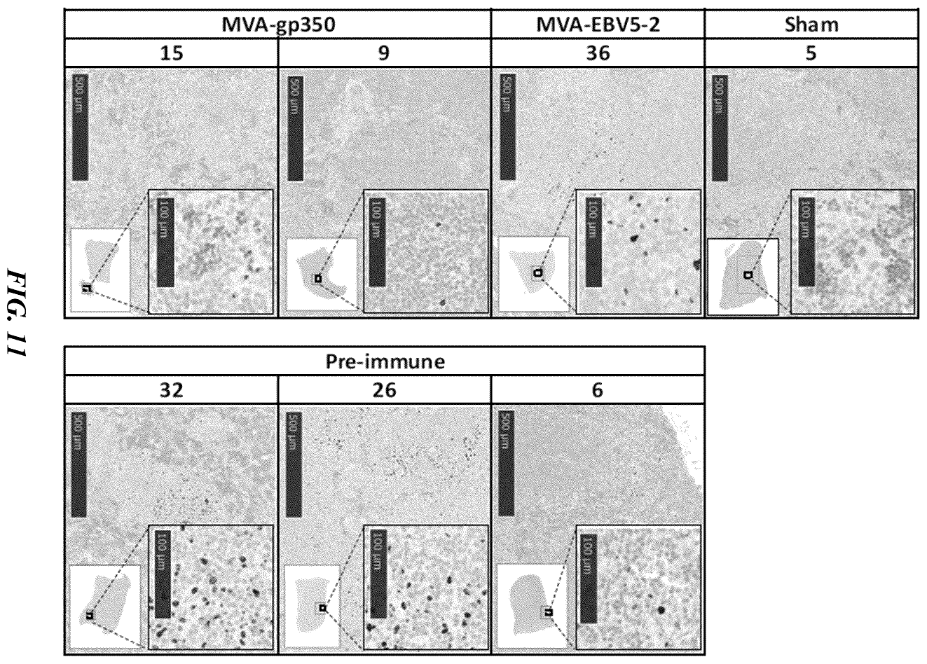

To assess whether MVA-EBV5-2 antibodies could prevent EBV infection in vivo, vaccine efficacy studies were performed in NSG mice engrafted with human CD34+ hematopoietic stem cells (NSG huMice). In two independent studies, NSG huMice were passively immunized with sera from immunized rhesus macaques and then challenged with either a low (FIG. 7A) or a high dose (FIG. 8A) of EBV. Before beginning the studies, mice from each huMice cohort were randomized into each treatment group, MVA-EBV5-2, MVA-gp350, Pre-immune or Sham, using a balanced allocation randomization algorithm to ensure that the mean percentage of human CD45-positive (% hCD45+) lymphocytes in the circulating blood of each animal was balanced across groups (FIG. 7B and 8B). Individual mouse information and respective % hCD45+ lymphocyte values assessed by flow cytometry (FIG. 9A) are listed in Table 1 and 2 for the low and high EBV dose challenge studies, respectively. Animals in groups MVA-EBV5-2 and MVA-gp350 were passively immunized with Day 56 pooled serum from immunized rhesus macaques, while the Pre-immune group received Day −7 pooled serum (negative control). After 12 hours, animals were challenged with either 5×10³ Raji IU (low dose, FIG. 7A) or 5×10⁴ Raji IU (high dose, FIG. 8A) of EBV. Sham group was neither immunized with sera nor challenged with EBV. The challenge timepoint was chosen based on a preliminary serum kinetics study (FIG. 9B), where rhesus IgG against EBV gp350 reached a maximum and stable level in NSG huMice serum 12-hours post-intraperitoneal passive immunization (FIG. 9C). Moreover, an intraperitoneal inspection of the animals at each final point showed no non-absorbed rhesus macaque sera after 12 hours. In both studies, EBV infection was assessed only in mice that reached the experimental endpoint and on samples harvested at death point when available for mice that did not finish the study (Table 1 and 2).

TABLE 1
Individual Mouse information and respective % hCD45+ lymphocyte values

| | | | | | BALF5 | BALF5 | |
|---|---|---|---|---|---|---|---|
| | | | | | copies/μg | copies/μg | Spleen |
| | Animal | | % hCD45+ | | of blood | of spleen | EBER-ish |
| Treatment | ID | Sex | lymphoocytes | Death | DNA | DNA | rating |
| MVA-EBV5-2 | 6 | F | 91.35 | Day 36 | ND | ND | ND |
| sera | 8 | F | 63.9 | At | 0.0 | 0.0 | − |
| % hCD45+ | | | | endpoint | | | |
| average: 79.78 | 10 | F | 77.4 | Day 53 | 0.0 | 0.0 | − |
| | 11 | F | 92.55 | Day 22 | ND | ND | − |
| | 17 | M | 85.15 | At | 0.0 | 0.0 | − |
| | | | | endpoint | | | |
| | 20 | M | 77 | Day 25 | 0.0 | 0.0 | − |
| | 22 | M | 71.1 | At | 0.0 | 7.9 | − |
| | | | | endpoint | | | |
| MVA-gp350 | 4 | F | 85 | Day 44 | 1722.0 | 15521.7 | ++++ |
| sera | 5 | F | 87.35 | At | 0.0 | 0.0 | − |
| % hCD45+ | | | | endpoint | | | |
| average: 79.24 | 7 | F | 94.75 | Day 45 | 0.0 | 11.5 | − |
| | 9 | F | 76.5 | At | 2571.3 | 299286.2 | ++++ |
| | | | | endpoint | | | |
| | 15 | M | 72.55 | Day 9 | ND | ND | ND |
| | 19 | M | 59.3 | Day 53 | 0.0 | 0.00 | − |
| | 21 | M | 79.2 | At | 0.2 | 137.1 | + |
| | | | | endpoint | | | |
| Pre-immune | 1 | M | 91.3 | At | 120.8 | 14011.1 | ++++ |
| sera | | | | endpoint | | | |
| % hCD45+ | 12 | F | 69.35 | At | 0.0 | 0.00 | − |
| average: 81.20 | | | | endpoint | | | |
| | 13 | F | 86.75 | Day 11 | ND | ND | ND |
| | 14 | M | 82.5 | Day 8 | ND | ND | ND |
| | 16 | M | 72.35 | Day 47 | 0.0 | 24.5 | + |
| | 24 | F | 91.85 | At | 140.2 | 232.9 | + |
| | | | | endpoint | | | |
| | 25 | F | 74.3 | At | 0.0 | 57.5 | + |
| | | | | endpoint | | | |
| Sham | 3 | M | 21.55 | At | 0.0 | 0.0 | − |
| % hCD45+ | | | | endpoint | | | |
| average: 22.93 | 18 | M | 24.3 | At | 0.0 | 0.0 | − |
| | | | | endpoint | | | |

ND = not determined

TABLE 2
Individual Mouse information and respective % hCD45+ lymphocyte values

| | | | | | BALF5 | BALF5 | |
|---|---|---|---|---|---|---|---|
| | | | | | copies/μg | copies/μg | Spleen |
| | Animal | | % hCD45+ | | of blood | of spleen | EBER-ish |
| Treatment | ID | Sex | lymphoocytes | Death | DNA | DNA | rating |
| MVA-EBV5-2 | 7 | F | 63.0 | At | 0.0 | 13.6 | − |
| sera | | | | endpoint | | | |
| % hCD45+ | 13 | F | 60.5 | At | ND | 0.0 | − |
| average: 66.47 | | | | endpoint | | | |
| | 24 | M | 59.2 | Day 57 | 0.0 | 6.8 | − |
| | 33 | F | 85.2 | At | 0.0 | 0.0 | − |
| | | | | endpoint | | | |
| | 36 | M | 36.3 | At | 66.7 | 2345.6 | +++ |
| | | | | endpoint | | | |
| | 41 | F | 91.7 | At | 0.0 | 0.0 | − |
| | | | | endpoint | | | |
| | 47 | F | 69.4 | At | 0.0 | 0.0 | − |
| | | | | endpoint | | | |
| MVA-gp350 | 9 | M | 81.5 | Day 27 | 880.0 | 5487.5 | +++ |
| sera | 10 | M | 51.2 | Day 11 | ND | ND | ND |
| % hCD45+ | 11 | M | 64.2 | Day 27 | ND | 3.0 | − |
| average: 66.40 | 12 | M | 95.2 | Day 25 | ND | ND | ND |
| | 15 | F | 46.9 | At | 20.0 | 735.4 | + |
| | | | | endpoint | | | |
| | 21 | F | 60.0 | At | 0.0 | 0.0 | − |
| | | | | endpoint | | | |
| | 44 | M | 65.8 | At | 70.0 | 547.0 | − |
| | | | | endpoint | | | |
| Pre-immune | 6 | F | 59.4 | At | 20.0 | 4648.9 | ++ |
| bleed sera | | | | endpoint | | | |
| % hCD45+ | 25 | M | 61.7 | At | 0.0 | 0.0 | − |
| average: 68.41 | | | | endpoint | | | |
| | 26 | M | 51.1 | At | 4026.0 | 34566.3 | ++++ |
| | | | | endpoint | | | |
| | 31 | F | 83.4 | Day 26 | ND | 6027.7 | ND |
| | 32 | F | 62.9 | At | 0.0 | 468.0 | +++ |
| | | | | endpoint | | | |
| | 40 | F | 70.1 | At | 0.0 | 24.7 | − |
| | | | | endpoint | | | |
| | 43 | M | 90.3 | Day 27 | 0.0 | 5.6 | − |
| Sham | 5 | F | 62.0 | At | 0.0 | 0.0 | − |
| % hCD45+ | | | | endpoint | | | |
| average: 60.53 | 8 | F | 80.4 | At | 0.0 | 0.0 | − |
| | | | | endpoint | | | |
| | 19 | M | 57.8 | At | 0.0 | 0.0 | − |
| | | | | endpoint | | | |
| | 23 | F | 61.5 | At | 0.0 | 0.0 | − |
| | | | | endpoint | | | |
| | 39 | F | 65.8 | At | 0.0 | 0.0 | − |
| | | | | endpoint | | | |
| | 42 | M | 35.7 | At | 0.0 | 0.0 | − |
| | | | | endpoint | | | |

In the low dose study, the mice were monitored for 56 days, after which the mice were euthanized, and blood and spleens were collected to assess infection outcomes (FIG. 7A). Viremia (FIG. 7C) and spleen infection (FIG. 7D) were measured by qPCR against the EBV BALF5 gene. Moreover, spleen infection was assessed by performing EBV-encoded RNA (EBER) in situ hybridization (FIG. 7E and 10). While 2/6 and ⅖ animals in the MVA-gp350 and Pre-immune groups displayed detectable viremia (FIG. 7C), respectively, no mice in the MVA-EBV5-2 group was positive for EBV in the blood. Reduced infection was observed in the spleens by qPCR (FIG. 7D), where only ⅕ animals in the MVA-EBV5-2 group displayed infection, with a low number of EBV DNA copies; meanwhile 4/6 and ⅘ mice presented higher infection levels in MVA-gp350 and Pre-immune groups, respectively. By EBER staining (FIG. 7E and 10), no positive animals were detected in the MVA-EBV5-2 groups, while 3/6 and 4/6 were identified in MVA-gp350 and Pre-immune groups, respectively. The perimeter and area of collected spleens were not significantly different between groups (FIGS. 7F and 7G).

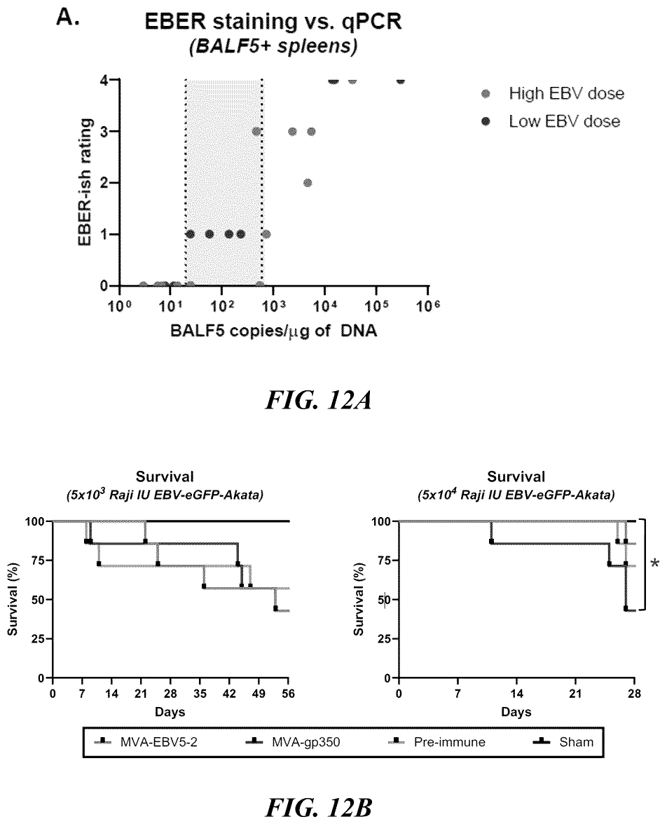

In the high dose EBV challenge study, in which a 10-fold higher EBV dose was used compared to the first cohort, the length of this study was reduced from 56 days to 28 days to ensure animal welfare (FIG. 8A), guided by periodic health evaluation of the animals. At the endpoint, blood and spleens were collected and the presence of EBV was evaluated by qPCR (FIG. 8C and 8D) and EBER in situ hybridization (FIG. 8E and 11) as before. Viremia evaluation (FIG. 8C) resulted in the detection of EBV DNA copies in ⅙ animals in the MVA-EBV5-2 group, ¾ animals in the MVA-gp350 group and 2/6 in the Pre-immune group. In the spleen, EBV DNA was detected in a higher number of mice in all treatment groups when compared to animals in the low EBV dose challenge study (FIG. 7D versus FIG. 8D), possibly owing to the higher dose of EBV used in this case. However, even at this high dose (FIG. 8D), the number of animals that displayed spleen infection was reduced in the MVA-EBV5-2 group ( 3/7), compared to the MVA-gp350 (⅘) and Pre-immune ( 6/7) groups. As in the low dose study, the spleen evaluation by EBER staining (FIG. 8E and 11) resulted in the detection of a lower number of EBV positive animals, 1/7 in the MVA-EBV5-2 group, ⅖ in the MVA-gp350 group and 3/6 in the Pre-immune group. As such, spleen qPCR and EBER staining for EBV detection was compared (FIG. 12A). Results showed that between 2×10¹ and 6×10² EBV copies/μg DNA as detected by qPCR, EBER staining can fail in the detection of EBV positive samples, with no detection under that range; on the other hand, qPCR was able to detect as few as 3 EBV copies/μg DNA, suggesting that qPCR can detect EBV infection with a higher sensitivity; future studies should use EBER staining as a complimentary technique to qPCR in detecting EBV infection. Regardless, EBER staining results support the previous observations that MVA-EBV5-2 animals displayed lower levels of infection than control groups. The perimeter and area of collected spleens were not significantly different between groups (FIGS. 8F and 8G).

Finally, regarding survival, mice in all groups except for Sham showed a general decline in health as the experiment progressed for both studies (FIG. 12B). No differences between the groups were detected, except between the MVA-gp350 and Sham groups in the high-dose study, but whether this effect is real and not due to other inherent health issues in the mice is unclear.

### Methods

The following methods were used to produce the results in the foregoing examples.

### Cell Lines and Viruses

All cell lines were incubated at 37° C. in the presence of 5% CO₂ and grown in media supplemented with 10% FBS, 1% L-glutamine, 2% penicillin-streptomycin unless otherwise noted, and were tested for mycoplasma contamination. BHK-21 are Syrian golden hamster kidney cells and were grown in DMEM media. AGS-Akata-EBV-eGFP are human female gastric adenocarcinoma cells harboring EBV in which the thymidine kinase (TK) gene has been replaced with a neomycin and GFP cassette (Akata-EBV-eGFP,²⁷); they were grown in DMEM/F-12 media additionally supplemented with 500 μg/ml G418. HEK-293 are human female embryonic kidney cells and were grown in DMEM media. Raji are human male Burkitt lymphoma cells and were grown in RPMI media. ExpiCHO-S are Chinese hamster ovary cells adapted to serum-free suspension culture and were grown in ExpiCHO Expression Medium.

Akata-EBV-eGFP virus was produced from AGS-Akata-EBV-eGFP cells, as described in Method Details. Recombinant MVA virus expressing EBV gp350, gB, gp42 and gHgL or gp350 alone was constructed via bacterial artificial chromosome (BAC) technology from MVA-BAC-TK, and reconstituted/expanded in BHK-21 cells as described in Method Details. MVA-BAC-TK harbors the MVA genome with the BAC pBeloBAC11 (GenBank: U51113) inserted at the position of the TK gene together with a GFP expression cassette. MVA-BAC-TK in GS1783 bacteria was developed from the MVA 1974/NIH clone 1. MVA-BAC-TK has been previously described⁶¹, as has GS1783 bacteria⁶².

### Mice

Female and male BALB/c mice aged 8-10 weeks purchased from Charles River Laboratories (Strain #028) were used for vaccine immunogenicity studies.

Female and male NOD.Cg-Prkdc^{scid pk II}2rg^{tm1Wjl}ISzJ (NSG) obtained from an ongoing NSG colony at the Beckman Research Institute of City of Hope Animal Resources Center, originally purchased from The Jackson Laboratory (Strain #005557; RRID: IMSR_JAX: 005557), were used for vaccine efficacy studies. Prior to vaccine efficacy studies, 3-5-week-old NSG mice were engrafted with CD34+ human hematopoietic stem cells to generate humanized mice as described in Method Details.

All mice were housed at BSL2 facilities in the Animal Resources Center of Beckman Research Institute of City of Hope, with free access to food and water in a 12:12 light: dark cycle. The animal facilities are accredited by the Association for Assessment and Accreditation of Laboratory Animal Care (AAALAC) and conform to the Institute for

Laboratory Animal Research Guide for the Care and Use of Laboratory Animals. All mouse procedures were performed in accordance with approved Beckman Research Institute of City of Hope Institutional Biosafety Committee and Institutional Animal Care and Use Committee protocols.

### Rhesus Macaques

Female and male Indian-origin rhesus macaques (Macaca mulatta) aged 9-24 years were leased from and housed at the Oregon National Primate Research Center (ONPRC) and used for vaccine immunogenicity studies. Animals were housed in specific-pathogen-free facilities and periodically tested for rhesus lymphocryptovirus infection. The ONPRC is an AAALAC-accredited research facility and conforms to National Institutes of Health guidelines on the ethical use of animals in research. All rhesus macaque procedures were performed in accordance with approved ONPRC IACUC and Beckman Research Institute of City of Hope IBC and IACUC protocols.

### Plasmids, Recombinant DNA and Oligonucleotides

All pCAGGS plasmid cloning and synthesis were performed by Genewiz at Azenta Life Sciences, using pCAGGS parental plasmid that has been previously described ⁹⁸. mH5-Kan-gp350-2A-gB-pCAGGS plasmid contains a bi-cistronic expression cassette that codes for full-length gp350 (GenBank: CAD53417.1) and gB (GenBank: CAD53463.1), interspersed by a 2A autocleavable peptide sequence. 2A peptides and associated sequences have been previously described^{48,64}. mH5-Kan-gp42-2A-gL-2A-gH-pCCAGS plasmid contains a tri-cistronic expression cassette that codes for full-length gp42 (GenBank: GenBank: CAD53422.1), gL (GenBank: CAD53428.1), and gH (GenBank: CAD53450.1), interspersed by 2A autocleavable peptide sequences. Expression cassettes for both plasmids are under the control of the modified H5 (mH5) promoter⁶³, which is followed by a I-Scel restriction enzyme site and the Kanamycin resistance (Kan R) gene flanked by two 40bp-duplication sequences, preceding the glycoprotein genes.

EBV-gp42-His-Avi-PTT3, EBV-gH-His-Avi-PTT3, and EBV-gL-His-Avi-PTT3plasmids coding for EBV gp42 (a.a. 33-223, GenBank: AFY97939.1), gH (a.a. 19-679, GenBank: AFY97969.1), and gL (a.a. 24-137, Genbank: AFY97944.1), respectively, have been previously described²⁸.

PTT3 plasmid cloning and synthesis for rhLCV-gp350-ecto-His-Avi-PTT3 were performed by Genewiz at Azenta Life Sciences, using PTT3 parental plasmid that has been previously described^{99,100}. rhLCV-gp350-ecto-His-Avi-PTT3 plasmid contains an expression cassette that codes for the rhLCV gp350 ectodomain (a.a. 1-739, NCBI Reference sequence: NC_006146.1, Gene ID: 2949805) fused to a 6xHis-Avidin tag.

Synthesis for all primers and BALF5 FAM-labeled probe and gBlock was performed by Integrated DNA Technologies.

### Generation of Recombinant MVA Vectors

Recombinant MVA vectors were generated by standard methods, using homologous recombination techniques. To generate MVA-EBV5-2, the glycoprotein expression cassettes in mH5-Kan-gp350-2A-gB-pCAGGS and mH5-Kan-gp42-2A-gL-2A-gH-pCCAGS plasmids were amplified using site-specific primers for homologous recombination that add 50-bp duplications of the desired MVA insertion site at each amplicon end, en passant 69R/70L and 64L/65L primer pairs, respectively. Two rounds of en passant mutagenesis were subsequently carried out in GS1783 bacteria harboring MVA-BAC-TK⁶², which resulted in the insertion of expression cassettes coding for gp350-2A-gB and gp42-2A-gL-2A-gH into the 69R/70L and 64L/65L MVA genomic sites, respectively, and removal of Kan R gene from each. To generate MVA-gp350, the sequence for mH5-Kan together with the gp350 ectodomain (a.a. 1-864) was first amplified from the mH5-Kan-gp350-2A-gB-pCAGGS plasmid using a site-specific primer for homologous recombination on the 5′ end (en passant Del3 forward primer), and a primer that adds a 6xHis tag on the 3′ end (gp350 ectodomain 6× tag Reverse primer). Then, the amplicon was further amplified with site-specific primers for homologous recombination on both ends (en passant Del3 primer pairs). One round of en passant mutagenesis in GS1783 bacteria harboring MVA-BAC-TK proceeded as above, which resulted in the insertion of the expression cassette coding for gp350-ectodomain-6xHis in the Del3 MVA genomic site, and removal of Kan R gene.

Both MVA-EBV5-2 and MVA-gp350 were reconstituted by transfecting respective purified recombinant DNA generated in GS1783 bacteria into BHK-21 cells previously seeded in a 6-well plate (1 μg DNA: 4 μg polyethyleneimine [Sigma Aldrich] per well). Four hours after transfection, fowlpox virus was added to each transfected well. The next day, media was replaced with growth media, and the cells subsequently monitored for eGFP expression, since the MVA-BAC-TK backbones contain an eGFP marker. In the next weeks, cells were maintained and grown until achieving 90-100% eGFP expression (full infection) in 18-20 150 mm cell culture dishes. To isolate the viruses, cells were harvested using a cell scraper, and then pelleted by centrifugation at 335xg speed at 4° C., for 20 min. Subsequently, cell pellets were frozen and thawed three times, and then sonicated for 4min. Resulting pellets were resuspended in Opti-MEM and centrifuged at 4300×g, at 4° C. for 10 min. The resulting supernatant, containing the virus (passage 0 [p0]), was then aliquoted and stored at −80° C. until use.

For animal experiments, viruses were grown and expanded in BHK-21 cells for large-scale production, and purified via ultracentrifugation through a sucrose cushion as described in^{101,102}. Resulting virus stocks were titrated in BHK-21 cells as described in ¹⁰³ and in “Flow cytometry-based virus titrations” below, using eGFP as a marker for infection.

### Genetic and Translational Stability Assessment of MVA-EBV5-2

To assess genetic and translational stability of MVA-EBV5-2, serial viral passaging was performed in BHK-21 cells. PO stock was used to infect five 150 mm dishes of BHK-21 cells. Once infection had spread and 90-100% of the cells expressed eGFP, cells were processed for virus isolation as described above, generating p1. The process was repeated up to p10, and additional infected cells were harvested at each passage for DNA isolation and immunoblot analysis.

To assess genetic stability of the inserted expression cassette, DNA from infected cells at each passage was isolated using the Dneasy Blood & Tissue Kit (QIAGEN) according to manufacturer's instructions. PCR was first used to assess the size of the two inserted glycoprotein expression cassettes by amplifying the sequences at the 69R/70L and 64L/65L MVA genomic sites. Resulting PCR products were analyzed by gel electrophoresis on an ethidium bromide 1% agarose gel. Additionally, p0 and p10 DNA was prepared and submitted for full genome PacBio sequencing to the City of Hope Integrative Genomics Core, according to the Core's instructions.

To assess translational stability of the virus, the isolated viruses were used to infect BHK-21 cell and assess expression of each individual glycoprotein via flow cytometry analysis, as described in “Flow cytometry-based assessment of glycoprotein expression in MVA vector-infected cells”. Additionally, total protein was isolated from the collected infected cells and processed for immunoblot as described in “Immunoblot-based assessment of glycoprotein expression in MVA vector-infected cells” to detect EBV gp350 and gp42.

### Recombinant Glycoproteins and Antibodies

Recombinant EBV gp350/220 ectodomain (a.a.4-863) is available commercially (Immune Tech Corp.). Recombinant EBV gB (a.a. 23-683, GenBank: AFY97983.1) has been previously described²⁸. Recombinant gp42gHgL (gH: a.a. 1-679, UniProtKB/Swiss-Prot: Q3KSQ3.1; gL: a.a. 1-137, UniProtKB/Swiss-Prot: P03212.1; gp42: a.a. 34-223, UniProtKB/Swiss-Prot: POC6Z5.1) has been previously described²⁹. Additional recombinant gp42gHgL (gp42: a.a. 33-223, GenBank: AFY97939.1; gH: a.a. 19-679, GenBank: AFY97969.1; gL: a.a. 24-137, Genbank: AFY97944.1) was produced and purified by GenScript, by co-transfection of EBV-gp42-His-Avi-PTT3, EBV-gH-His-Avi-PTT3, and EBV-gL-His-Avi-PTT3 plasmids in mammalian cells.

Recombinant rhLCV gp350 ectodomain (a.a. 1-739, NCBI Reference sequence: NC_006146.1, Gene ID: 2949805) was produced in-house via the ExpiCHO Expression System in ExpiCHO-S cells (ThermoFisher Scientific) according to the kit's instructions, using the rhLCV-gp350-ecto-His-Avi-PTT3 plasmid. Resulting protein was purified via ÄKTA pure chromatography system (Cytiva).

The murine antibodies F-2-1, CL40 and E1D1 against EBV gp42, gH and gL, respectively, have been previously described²⁵⁻²⁷. The murine antibody HB5 against EBV gp350 has been previously described³⁰, and was produced in-house at the Ogembo laboratory as described³⁰ and purified at the X-Ray Crystallography and Macromolecular Characterization Core at the Beckman Research Institute of City of Hope. Similarly, the murine antibody 15C8 against EBV gp42 was generated by the traditional mouse hybridoma method as described for HB5³⁰, and produced and purified as above for HB5. The human antibody AMMO5 against gB has been previously described²⁸. Rat hybridoma 19C2 against vaccinia B5R has been previously described¹⁰⁴.

### Flow Cytometry-Based Assessment of Glycoprotein Expression in MVA Vector-Infected Cells

BHK-21 cells previously seeded in a 48-well plate were infected with empty MVA or MVA-EBV5-2 viruses in triplicate per each glycoprotein/antibody to be tested and incubated overnight. Cells from each well were harvested and washed with PBS twice, after which they were incubated for 1 hour at room temperature with glycoprotein-specific primary antibody diluted in PBS (5 μg/ml). Cells were washed with PBS twice and were then incubated for 1 hour at room temperature with corresponding Alexa Fluor-647-conjugated secondary antibody diluted in PBS ( 1/2000). Cells were washed twice with PBS, and then fixed in 1% paraformaldehyde (PFA, Electron Microscopy Sciences). Stained cells were subsequently analyzed via flow cytometry in a BD Accuri C6 equipment (BD). Obtained data was analyzed using FlowJo software and results were plotted using GraphPad Prism software.

### Immunoblot-Based Assessment of Glycoprotein Expression in MVA Vector-Infected Cells

Uninfected BHK-21 cells, BHK-21 cells that had been infected overnight with empty MVA or MVA-EBV5-2 viruses, or infected cells obtained from serial passaging of MVA-EBV5-2 were harvested using a cell scraper, and then pelleted by centrifugation at 335×g speed at 4° C., for 10 min. Pellets were washed twice with cold PBS, and then lysed with Mammalian Cell Lysis Buffer treated with Pierce Protease Inhibitor, according to manufacturer's instructions. The resulting cell lysates were prepared for SDS-PAGE by boiling the cell lysate with an appropriate volume of 6× reducing Laemmli SDS sample buffer at 100° C. for 5 min, and loading the samples on a Bolt 4-12%, Bis-Tris Plus gel. After gel electrophoresis, proteins were transferred to a nitrocellulose membrane via the iBlot 2 system, and subsequently incubated in blocking buffer (3% BSA in 0.1% Tween-20 PBS) for 1 hour at room temperature, shaking. The membranes were then incubated overnight at 4° C., shaking, with primary antibody diluted in blocking buffer, 10 μg/ml for purified HB5 or 19C2 antibodies, and a 1:10 dilution for 19C2 hybridoma supernatant. Membranes were washed three times with wash buffer (0.1% Tween-20 PBS) five minutes each time, shaking. They were then incubated for 1 hour at room temperature, shaking, with secondary antibody diluted in blocking buffer, 1/2000 for HRP-conjugated anti-mouse IgG and 1/2500 for HRP/conjugated anti-rat IgG. The membranes were then washed as before and were briefly incubated with SuperSignal West Pico PLUS Chemiluminescent Substrate after which they were immediately imaged in a PXi Multi-Application Gel Ima7ging System.

### Flow Cytometry-Based Virus Titrations

For Akata-EBV-eGFP titrations, Raji cells or HEK-293 cells previously seeded in 96-well plates or 48-well plates, respectively, were infected with various volumes of Akata-EBV-eGFP in triplicate and incubated overnight. Cells from each well were harvested and washed with PBS twice, after which they were fixed in 1% PFA. Cells were subsequently analyzed for eGFP expression via flow cytometry in a BD Accuri C6equipment or NovoCyte Quanteon 4025. Obtained data was analyzed using FlowJo software and results were plotted using GraphPad Prism software. Raji infectious units (Raji IU) per volume were calculated for Raji titrations by using the following formula, described in ¹⁰⁵: Raji IU/volume of virus=(number of cells at time of infection×percent of eGFP-positive cells)/volume of inoculum. The formula was applied to each datapoint in the linear range of each infection curve obtained, and values averaged to obtain Raji IU/volume.

Titration of MVA viruses in BHK-21 cells was performed as described above for Akata-EBV-eGFP titrations in HEK-293 cells. Flow cytometry analysis of eGFP expression was performed in BD Accuri C6 equipment and obtained data was analyzed using FlowJo software. To calculate relative IU (RIU) per volume, the following formula was applied, as described in ¹⁰³: for >30% eGFP-positive cells, RIU/volume of virus=cell number at the time of infection×[−LN (1−[p/100])]×(viral dilution factor/volume of inoculum); for <30% eGFP-positive cells, RIU/volume of virus=cell number at the time of infection×(p/100)×(viral dilution factor/volume of inoculum). The formula was applied to a single datapoint in the linear range of the infection curve obtained to calculate the RIU/volume.

### EBV-Reporter Virus Production

To produce GFP reporter virus (Akata-EBV-eGFP), AGS-Akata-EBV-eGFP cells were cultured in 15-cm dishes until reaching ˜90% confluency. Growth media was replaced with DMEM/F-12 containing 10% FBS, 2% pen-strep, 33 ng/ml 12-O-tetradecanoylphorbol-13-acetate and 2 mM sodium butyrate to induce viral lytic replication, and cells were subsequently incubated for 24 hours. After the incubation, the induction media was replaced with growth media without G418, and cells were incubated for an additional 5-7 days. Cell supernatants were collected and centrifuged at 4300×g speed for 90 min, at 4° C. Resulting supernatants were filtered to remove cell debris (0.8 μm), followed by two sequential ultra-centrifugations in Beckman-Coulter type 19 rotors at 38,200×g for 90 min at 4° C. to concentrate the virus. Resulting pellets at each centrifugation were resuspended in Opti-MEM, pooled, and stored at −80° C. until use.

### Immunizations in BALB/c Mice

8-10-week-old female BALB/c mice were immunized intraperitoneally on Day 0, Day 28, and Day 56 for a total of three times with 5x107 RIU of MVA-EBV5-2, MVA-gp350, or empty MVA, or with 5x10⁴ Raji IU of UV-inactivated EBV (UV-EBV) (n=8/group). Blood was collected from the submandibular vein 7 days prior to primary immunization, and on Days 21, 49, 84, and 119, after which the animals were terminally bled on Day 182 via cardiac puncture. Serum was separated from whole blood by collecting blood in serum collection tubes containing a clotting activator, and centrifuging collection tubes at 10,000×g for 5 min. This experiment was repeated identically in 8-10-week-old male BALB/c mice, with the exception that mice were terminally bled on Day 119.

Measurement of rhLCV Seroreactivity in Rhesus Macaques by ELISA

Serum anti-rhLCV-gp350 IgG in rhesus macaques prior to immunization was measured via ELISA using soluble recombinant rhLCV gp350 as target antigen. 96-well Nunc MaxiSorp flat-bottom microplates were coated overnight at 4° C. with 100 ng/well of the target antigen in PBS (100 μl at 1 μg/ml). Plates were blocked with 200 μl blocking buffer (3% BSA in 0.1% Tween-20 PBS) for 1 hour at room temperature, shaking. 100 μl of serum from individual animals diluted 1/100 in PBS were then added to the plates in duplicates. Plates were incubated for 2 hours at room temperature, shaking, followed by incubation for 1 hour at room temperature, shaking, with 100 μl of HRP-conjugated anti-rhesus IgG diluted 1/5000 in PBS. Between each step, plates were washed three times with 300 μl wash buffer (0.1% Tween-20 PBS). Lastly, plates were incubated for 20 minutes with 100 μl of ABTS 2-Component Microwell Peroxidase Substrate. Reactions were stopped with 100 μl of ABTS Peroxidase Stop Solution, and the OD of the reactions were read at 405 nm with a Filter Max F3 microplate reader. Obtained data was analyzed and plotted using GraphPad Prism software.

### Immunizations in Rhesus Macaques

9-24-year-old female and male Indian-origin rhesus macaques were immunized intramuscularly on Day 0 and Day 28 for a total of two times with 1×10⁸ RIU of MVA-EBV5-2 or MVA-gp350, or with 1×10⁵ Raji IU of UV-EBV (n=5/group). Blood and saliva were collected under ketamine sedation 7 days prior to primary immunization, and on Days 21, 56, 84, 112, 140, 168 and 196. Blood was collected by venipuncture via the saphenous vein; serum was separated from whole blood by collecting blood in serum collection tubes containing a clotting activator, and centrifuging collection tubes at 1000×g for 10 min. Saliva was collected using saliva collection swabs by placing the swab in both the left and right cheeks of the macaques. The swabs were left in the mouth for 1-3 min to allow production and absorption of saliva, after which they were placed back in the swab container, and 1 mL PBS added. Swabs were subsequently spun down in their containers at 1000×g for 10 min.

### Measurement of Glycoprotein-Specific Antibodies by ELISA

Glycoprotein-specific IgG in immunized mice (total IgG, IgG1 or IgG2a) was measured via ELISA using soluble recombinant gp350, gB, and gp42gHgL as target antigens. Ninety-six-well Nunc MaxiSorp flat-bottom microplates were coated overnight at 4° C. with 25 ng/well of the target antigen in PBS (50 μl at 0.5 μg/ml). Plates were blocked with either 100 μl milk blocking buffer (5% nonfat milk in PBS, total IgG) or BSA blocking buffer (IgG1 and IgG2a) for 1 hour at room temperature, shaking. Equal amounts of serum from each animal for each treatment group and timepoint were pooled, diluted in 1/900 in 1% milk in PBS (total IgG), and added to the plates in triplicate (50 μl). Alternatively, individual mouse serum samples were assessed at a 1/900 dilution in PBS (IgG1 and IgG2a). Plates were incubated for 2 hours at room temperature, shaking, followed by incubation for 1 hour at room temperature, shaking, with 50 μl of either HRP-conjugated anti-mouse IgG ( 1/2000 dilution in 1% nonfat milk in PBS), IgG1 ( 1/2000 dilution in PBS), or IgG2a ( 1/10,000 dilution in PBS). Between each step, plates were washed three times with 300 μl wash buffer. Lastly, plates were incubated for 20 min with 100 μl of ABTS 2-Component Microwell Peroxidase Substrate (LGC SeraCare, Milford, MA, USA). Reactions were stopped using 100 μl of ABTS Peroxidase Stop Solution (LGC SeraCare), and the OD of the reactions was read at 405 nm with a Filter Max F3 microplate reader (Molecular Devices). Obtained data was analyzed and plotted using GraphPad Prism software.

Serum and saliva glycoprotein-specific IgG of immunized rhesus macaques were measured via ELISA using soluble recombinant gp350, gB, and gp42gHgL as target antigens. Ninety-six-well Nunc MaxiSorp flat-bottom microplates were coated overnight at 4° C. with 50 ng/well of the target antigen in PBS (100 μl at 0.5 μg/ml for serum assays, 50 μl at 1 μg/ml for saliva assays). Plates were blocked with either 200 μl of milk blocking buffer or 100 μl of BSA blocking buffer for serum or saliva assays respectively, 1 hour at room temperature, shaking. One hundred μl of serum from individual animals diluted 1/100 in 1% nonfat milk in PBS, or 50 μl of undiluted saliva from individual animals were then added to the plates in duplicate. Plates were incubated for 2 hours at room temperature, shaking, followed by incubation for 1 hour at room temperature, shaking, with either 100 μl of HRP-conjugated anti-rhesus IgG diluted 1/5000 in 1% nonfat milk in PBS for serum assays, or 50 μl HRP-conjugated anti-rhesus IgG diluted 1/1000 in PBS for saliva assays. Between each step, plates were washed three times with 300 μl wash buffer. Lastly, plates were incubated for 20 min with 100 μl of ABTS 2-Component Microwell Peroxidase Substrate. Reactions were stopped with 100 μl of ABTS Peroxidase Stop Solution, and the OD of the reactions was read at 405 nm using a Filter Max F3 microplate reader. Obtained data was analyzed and plotted using GraphPad Prism software.

### Epithelial Cell Neutralization Assay

For epithelial cell neutralization assay using mouse sera, HEK-293 cells were seeded in a 48-well plate at a density of 50,000 cells/well and incubated overnight. The next day, Akata-EBV-eGFP virus, at a volume that yielded ˜10% infection, was incubated with serially diluted pooled immune mouse sera in a total of 100 μl of Opti-MEM for one hour. Following the incubation, the virus/sera mixture was added to each well in triplicates and incubated for another hour. Infection media was removed and 300 μl of growth media was subsequently added to each well, and the cells were incubated overnight. The next day, cells were processed for flow cytometry as described above for Akata-EBV-eGFP titration in HEK-293 cells. Percent neutralization values for every dilution were calculated by using the following formula: percent neutralization=100−(% eGFP-positive cells x 100/% eGFP-positive cells in negative control sample). Pooled serum from MVA-immunized mice was used as the negative control reference sample at each dilution.

For epithelial cell neutralization assay using rhesus macaque sera, HEK-293 cells were seeded in a 48-well plate at a density of 75,000 cells/well and incubated overnight. The next day, Akata-EBV-eGFP virus, at a volume that yielded ˜25% infection, was incubated with serially diluted individual rhesus macaque serum in a total of 100 μl of Opti-MEM for one hour. Following the incubation, the virus/sera mixture was added to each well in duplicates and incubated for another hour. 500 μl of growth media was subsequently added to each well, and the cells were incubated overnight. The next day, cells were processed for flow cytometry as described above for Akata-EBV-eGFP titration in HEK-293 cells. Percent neutralization for every animal at each dilution was calculated using the formula described above, using Pre-immune serum from each corresponding animal as the negative control reference sample. IC50 for each sample was calculated as the last serum dilution in which ≥50% neutralization was achieved.

### B Cell Neutralization Assay

For B cell neutralization assays using mouse sera, Raji cells were seeded in a 96-well plate at a density of 50,000 cells/well in 50 μl of Opti-MEM. The same day, Akata-EBV-eGFP virus, at a volume expected to yield 10-15% infection (real yield ˜5% infection), was incubated with serially diluted pooled immune mouse sera in a total of 50 μl of Opti-MEM per well for one hour at 37° C. Following the incubation, the virus/serum mixture was added to duplicate wells and incubated for another hour at 37° C. Two hundred-fifty ul of growth media was subsequently added to each well, and the cells were incubated overnight. The next day, cells were processed for flow cytometry as described above for Akata-EBV-eGFP titration in Raji cells. The percent neutralization for each dilution was calculated using the formula described above for HEK-293 cells.

For B cell neutralization assay using rhesus macaque sera, Raji cells were seeded in a 96-well plate at a density of 50,000 cells/well in 50 μl of Opti-MEM. The same day, Akata-EBV-eGFP virus, at a volume that yielded ˜5% infection, was incubated with serially diluted individual rhesus macaque serum in a total of 50 μl of Opti-MEM for one hour. Following the incubation, the virus/sera mixture was added to each well in duplicates and incubated for another hour. 250 μl of growth media was subsequently added to each well, and the cells were incubated overnight. The next day, cells were processed for flow cytometry as described above for Akata-EBV-eGFP titration in Raji cells. Percent neutralization for every animal at each dilution was calculated using the formula described above for HEK-293 cells, using Pre-immune serum from each corresponding animal as the negative control reference sample. IC50 for each sample was calculated as the last serum dilution at which ≥50% neutralization was achieved.

### Human Lymphocyte Engraftment of NSG Mice

To humanize NSG mice, 3-5-week-old NSG mice were engrafted with CD34+ human hematopoietic stem cells. Human stem cells were obtained from Advanced Bioscience Resources following regulatory guidelines and enriched for CD34+ hematopoietic stem cells using EasySep Human CD34 Positive Selection Kit II (STEMCELL) as per the manufacturer's instructions. Mice were then subjected to 240 cGY of whole-body radiation, followed by a retro-orbital injection of 1×10⁵ CD34-enriched human cells per mouse. 12 weeks post-engraftment, humanization in each mouse was assessed via flow cytometry. Blood was collected retro-orbitally from each mouse in EDTA-coated tubes, and subsequently incubated with 2 μl PE-conjugated anti-human CD45 antibody at 4° C. for 30 min in the dark. 2 ml red blood cell lysis buffer (Sigma Aldrich) was then added to lyse red blood cells and samples were incubated for 10 min in the dark at room temperature. Cell suspensions were centrifuged at 450×g for 5 min, supernatants removed, and cells were washed with 200 μl of PBS. Cell suspensions were then centrifuged at 600×g for 5 min, supernatants removed, and cells fixed in 2% PFA. Stained cells were analyzed via flow cytometry in LSRFortessa equipment (BD) to determine the percentage of human CD45 (hCD45) lymphocytes in every animal. Obtained data was analyzed using FlowJo software and results were plotted using GraphPad Prism software. NSG mice with successful human lymphocyte engraftment (humanized) are herein referred to as NSG huMice.

Rhesus Macaque Serum Kinetics in NSG huMice

NSG huMice mice (n=12) were immunized intraperitoneally with 500 μl of pooled Day 56 rhesus macaque UV-EBV-immune sera. Mice were then sequentially terminally bled by cardiac puncture at 3-, 6-, 12- and 24-hours post-immunization (n=3/timepoint); n=3 untreated mice were also terminally bled at the start of the experiment. The levels of gp350-specific rhesus IgG in the sera of NSG huMice were determined by ELISA at each timepoint, similarly to described above for sera of immunized rhesus macaques, with a few differences: plates were coated with 100 ng/well of target antigen (100 μl at 1 μg/ml), and mouse sera was diluted 1/50 in PBS. In addition, the intraperitoneal cavity of each mouse was inspected after euthanasia for the presence of undiffused rhesus macaque sera at each timepoint post-immunization.

EBV Challenge in NSG huMice

Two independent challenge experiments were performed. Prior to start of the studies, mice were sorted and randomly allocated into the different treatment groups using a balanced allocation algorithm according to the % hCD45 lymphocytes in each animal via WINPEPI software⁹⁷, to ensure similar distribution of humanization levels across treatment groups. In both experiments, mice were immunized intraperitoneally with 500 μl of pooled rhesus macaque sera from either Day −7 (all animals, Pre-immune), Day 56 MVA-EBV5-2 animals, or Day 56 MVA-gp350 animals (n=7/group). 12 hours post-immunization, animals were challenged intraperitoneally with Akata-EBV-eGFP. In the low-dose experiment, mice received 5x10⁵ Raji IU Akata-EBV-eGFP and were monitored for 56 days; n=2 additional mice were neither immunized nor challenged to serve as a Sham control. In the high-dose study, mice received 5×10⁶ Raji IU Akata-EBV-eGFP and were monitored for 28 days; n=6 additional mice were neither immunized nor challenged to serve as a Sham control. After the observation period, mice were terminally bled, and their spleens collected. Spleens were imaged using a standard smartphone camera, and the perimeter and area of each spleen were quantified from these images using IC Measure software. Spleens were divided in half, and each half was processed differently to assess infection outcomes. The first half was processed for immunohistochemistry and EBV-encoded small RNA (EBER) in situ hybridization (ish). Spleens were fixed in 10% formaldehyde, and then stored in 70% ethanol before submission for processing to the to the City of Hope Pathology Solid Tumor Core. The second half was processed along with blood for infection detection via qPCR as described in “Quantitative PCR analysis of human cells in humanized mice.”

### Quantitative PCR Analysis of Human Cells in Humanized Mice

To assess viremia and infection outcomes in challenged humanized mice, DNA was first isolated from collected blood and spleens using the DNeasy Blood & Tissue Kit according to manufacturer's instructions. For blood, 70 μl were processed per animal, and resulting DNA eluted in 50 μl of elution buffer. TaqMan qPCR reactions were then prepared using EBV BALF5-specific primers and probe. Each 25 μl reaction contained 1.25 μl of primer-probe mix (600 nM of each primer, 300 nM of FAM-labeled probe), 1.25 μl of TaqMan 20× VIC-labeled human RNase-P primer-probe mix, 12.5 μl of 2× QuantiTect Probe PCR Master Mix, and 10 μl of test sample (undiluted blood DNA or 1 μg of spleen DNA). To quantify BALF5 amplicons, a standard curve with known copy numbers was generated using a double-stranded DNA gBlock containing a partial sequence of the BALF5 gene, ranging from 10⁰ to 10⁸ BALF5 copies/μl. BALF5 copy numbers in each test sample were determined by interpolating from the standard curve. Reactions were first heated to 95° C. for 15 minutes, followed by 50 cycles of incubation at 95° C. for 15 seconds and 60° C. for 1 minute in a QuantStudio 3 Real-Time PCR System. For blood samples, resulting BALF5copy numbers were normalized according to each individual sample DNA concentration to present the results as EBV DNA copies/μg of DNA. Results were plotted using GraphPad Prism. For graphing purposes, all negative values based on the qPCR detection limit (<1 EBV DNA copies/ug of DNA) were assigned a value of 0.1.

### Quantification and Statistical Analysis

All statistical analyses were performed using GraphPad Prism software. For every case, the distribution of the scale variables was studied and analysis was performed using the appropriate model accordingly. In mouse immunogenicity assessments, differences in IgG responses between treatment groups on Day 49 and Day 84 were assessed via Tukey's multiple comparison test (single pooled variance) under a mixed-effects model. In rhesus macaque immunogenicity assessments, differences in IgG responses between treatment groups were assessed via Tukey's multiple comparison test (individual variances computed for each comparison) under a two-way ANOVA. Differences in IC50 between treatment groups for rhesus macaque neutralization assays were assessed via Dunn's multiple comparison test under a Kruskal-Wallis test. In NSG huMice studies, differences in % hCD45+ lymphocytes between groups were assessed via Tukey's multiple comparison test (single pooled variance) under an ordinary one-way ANOVA. Differences in EBV DNA copies (blood and spleen) between groups were assessed via Mann-Whitney test.

### Conclusion

EBV infection and its associated diseases remain a significant health burden, with no licensed prophylactic vaccine available despite four decades of vaccine research. Recently, EBV vaccine efforts have been shifting towards multivalent approaches that target multiple viral entry glycoproteins, as opposed to the previously dominant approach focused on a single entry glycoprotein, gp350⁶. The present technology describes the design, generation and characterization of an MVA-based vaccine, MVA-EBV5-2, that incorporates five EBV glycoproteins important for viral entry into diverse cell types: gp350, gB, gp42 and gHgL. After confirming that the expression cassettes coding for the five glycoproteins in MVA-EBV5-2 are genetically and translationally stable in 10 viral passages and expresses each target glycoprotein, the immunogenicity and efficacy of the vaccine was characterized in three animal models: BALB/c mice, rhesus macaques, and NSG huMice.

In immunized BALB/c mice and rhesus macaques, MVA-EBV5-2 elicited a robust serum IgG response against the target glycoproteins, as measured by ELISA. MVA-EBV5-2 elicited glycoprotein-specific IgG of both IgG2a and IgG1 subtypes, as opposed to MVA-gp350 control, which primarily elicited IgG1. This suggests that MVA-EBV5-2 elicited both Th1-and Th2-type immune responses. A balanced Th1/Th2 response implies that the vaccine can elicit both strong humoral immunity against EBV virions, as well as cytotoxic cellular immunity that could target EBV-infected cells should the virus escape Ab-mediated clearance^{71,72}. Additionally in macaques, MVA-EBV5-2-elicited IgG can be detected in the saliva of immunized animals, which has not been reported in previous EBV vaccine studies. Despite the potential protection that these IgG could provide in the oral cavity, exploring alternative immunization routes that target the mucosa will be key in further enhancing the salivary humoral response against this orally transmitted virus.

In the mouse experiments of this study, both MVA-EBV5-2 and MVA-gp350 sera outperformed UV-EBV sera in HEK-293 cells, with MVA-gp350 displaying slightly higher levels of neutralizing activity than MVA-EBV5-2 in Day 49 and Day 84 serum for female and male mice, respectively. EBV infection of HEK-293 cells is dependent on CR2/CD21¹³, a gp350 cellular receptor, and is thus expected to be susceptible to gp350-specific antibody-mediated EBV neutralization^{47,48}. In Raji neutralization, MVA-EBV5-2 sera outperformed MVA-gp350 sera in both female and male mice, suggesting that additional antibodies could be involved in neutralization of this cell line, as levels of serum gp350-specific IgG were similar between MVA-EBV5-2-and MVA-gp350-immunized mice. In the rhesus macaque experiment, MVA-EBV5-2 sera outperformed both MVA-gp350 and UV-EBV sera in neutralization assays of both cell lines.

In the absence of a readily accessible and truly representative in vivo EBV infection model, humanized mice are becoming increasingly popular for testing antibody and vaccine efficacy against EBV infection^{31,33-35,45,46,82-85}. The present examples use NSG huMice^{86,87} to test the protective efficacy of passively immunized MVA-EBV5-2 macaque immune sera against two dose levels of EBV. In both studies, in vivo results were consistent with in vitro results, as MVA-EBV5-2-immune sera provided superior protection against infection than MVA-gp350-immune sera. Previous EBV vaccines explored in humanized mice studies include gHgL-based nanoparticles⁸², gp350-based nanoparticles in combination with either gHgL- or gp42gHgL-based nanoparticles⁴⁵, and either gHgL or trivalent gB protein⁴⁶. Both nanoparticle studies, Malhi et al⁸² and Wei et al⁴⁵, used purified IgG from immunized animals rather than immune sera for passive immunization experiments, while the protein study, Cui⁴⁶, has been the only study before the present technology that used immune sera. In all three cases the experimental vaccines were able to reduce infection when compared to non-immune controls. Although not a vaccine study, Singh⁸⁴ compared the protective efficacy of the gHgL-specific neutralizing antibody, AMMO1, to the gp350-specific neutralizing antibody, 72A1, and found superior protection against infection in humanized mice passively immunized with AMMO1. Other antibody studies in humanized mice, although not including comparisons to gp350-specific antibodies, have also demonstrated the neutralizing potential of non-gp350-specific antibody glycoprotein targets^{31,33-35}.

### Example 5

### Generation and Characterization of MVA-EBV5-5 and Comparison to MVA-EBV5-2

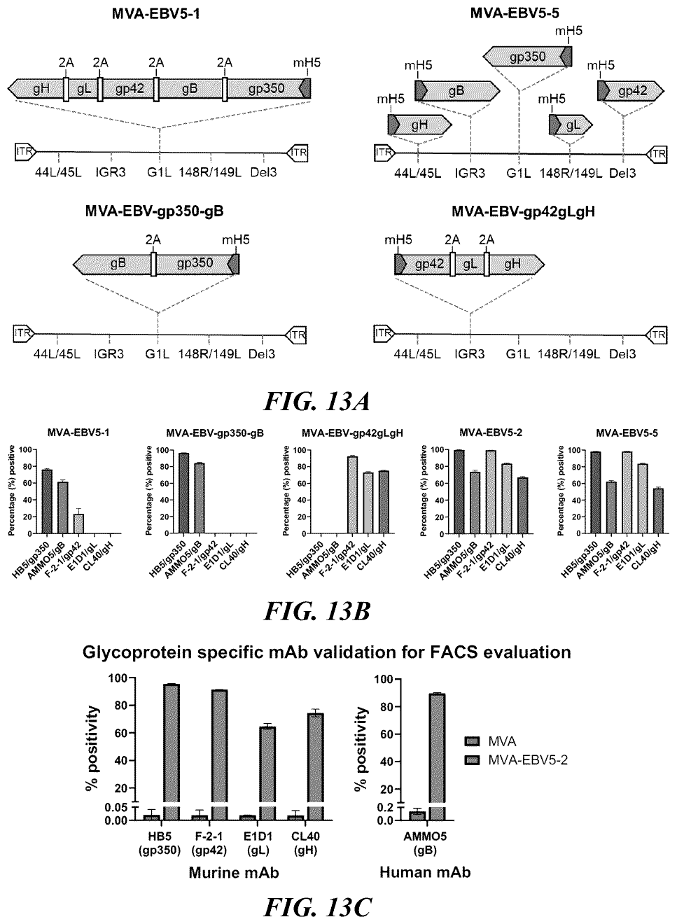

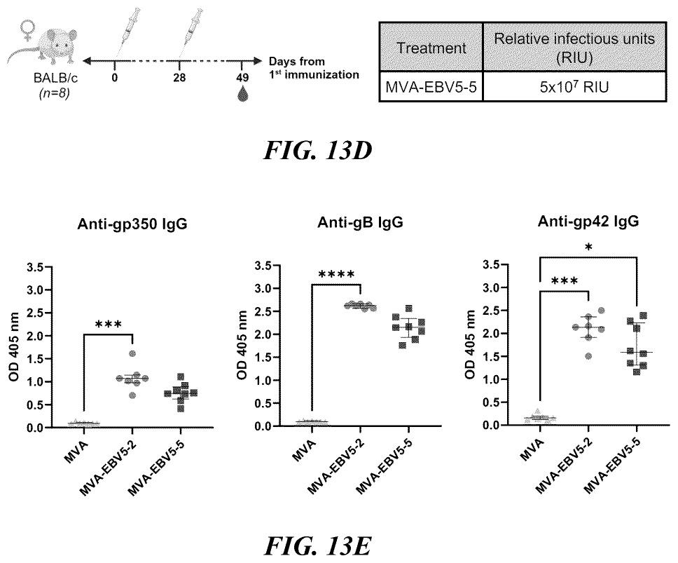

In the interest of designing a multivalent EBV vaccine incorporating five EBV glycoproteins involved in viral entry of B cells and epithelial cells, an eGFP-expressing MVA bacterial artificial chromosome (MVA-BAC-TK)⁶¹ was used to generate recombinant MVA vectors that incorporate gp350, gB, and gp42gHgL. First, a polycistronic glycoprotein expression cassette that codes for all five EBV glycoproteins in wildtype form was cloned into the MVA BAC via en passant mutagenesis, a homologous recombination-based cloning system⁶², generating MVA-EBV5-1 (FIG. 13A, top left). As mentioned above, two polycistronic glycoprotein expression cassettes that code for either gp42, gL, and gH (trivalent) or gp350 and gB (bivalent) were cloned in wildtype form into the MVA BAC (FIG. 1A). During this process, MVA vectors were also generated expressing the bivalent and trivalent glycoprotein expression cassettes contained within MVA-EBV5-2, MVA-EBV-gp350-gB and MVA-EBV-gp42gLgH (FIG. 13A, bottom left and right), which were subsequently used as controls in protein expression assessments. Finally, five expression cassettes were cloned coding for each glycoprotein individually into the MVA BAC (FIG. 13A, top right), generating MVA-EBV5-5. In all cases, each cassette codes for each corresponding glycoprotein(s) under the control of an mH5 promoter, a promoter that has been shown to stabilize transgenes in recombinant MVA constructs⁶³. To allow for individual glycoprotein release upon transcription, the glycoprotein sequences in all polycistronic cassettes are interspersed by unique self-cleaving 2A peptides⁶⁴.

To verify sequence fidelity before viral reconstitution, Sanger sequencing was performed for each expression cassette in the final recombinant MVA constructs. The resulting sequence analysis for MVA-EBV5-1 and MVA-EBV5-5 revealed full alignment with the expected sequence, except for the presence of Δ84bp (Δa.a.513-540) and Δ210bp (Δa.a.513-583) truncations in the gp350 ectodomain, respectively (data not shown). Given the presence of several 29-bp repetitive sequences in gp350, use of the en passant system can result in homologous recombination of these repetitive sequences, allowing the emergence of several truncated gp350 products. However, these truncations, including the Δ84bp and Δ210bp truncations, occur within the gp220 splice site, which has been excluded from other recent gp350-targeting vaccines^{45, 65, 66}, and do not overlap with any of the previously identified gp350 neutralizing epitopes³⁰; thus it is unlikely that they affect antigen immunogenicity. Similar Sanger sequencing results were obtained for MVA-EBV5-2, with the expression cassettes matching the expected sequence except for the Δ210bp (Δa.a.513-583) truncation in the gp350 ectodomain (data not shown). Sanger sequencing of MVA-EBV-gp350-gB and MVA-EBV-gp42gLgH yielded results that fully matched those of MVA-EBV5-2 (data not shown). Then the DNA was transfected for each recombinant MVA construct into MVA-permissive BHK-21 cells and reconstituted the viruses using helper fowlpox virus, generating passage 0 (p0) viruses (FIG. 1B).

Following reconstitution of p0 virus, surface glycoprotein expression was assessed for each recombinant MVA virus in infected BHK-21 cells, via flow cytometry (FIG. 13B and 13C). As expected, gp350 and gB expression were effectively detected on the surface of BHK-21 cells infected with MVA-EBV-gp350-gB, MVA-EBV5-2 and MVA-EBV5-5, while gp42, gL and gH were similarly detected on the surface of cells infected with MVA-EBV-gp42gLgH, MVA-EBV5-2 and MVA-EBV5-5. However, in comparison, gp350 and gB were detected on the surface of a lower percentage of MVA-EBV5-1-infected cells, and these cells also displayed minimal gp42 surface expression and non-detectable levels of gL or gH expression. After observation of low level of glycoprotein expression, which could be the result of the nature of the 2A-based polycistronic expression cassette¹⁰⁶, MVA-EBV5-2 and MVA-EBV5-5 became the focus instead of MVA-EBV5-1 as a potential multivalent vaccine candidates. Preliminary MVA-EBV5-5 immunogenicity experiments in BALB/c mice (FIG. 13D), when compared to MVA-EBV5-2 immunogenicity results described previously (FIGS. 2-3), led to the selection of MVA-EBV5-2 as the lead vaccine candidate, as MVA-EBV5-2 immunization led to higher levels glycoprotein-specific IgG overall (FIG. 13E). Thus, MVA-EBV5-2 was further characterized.

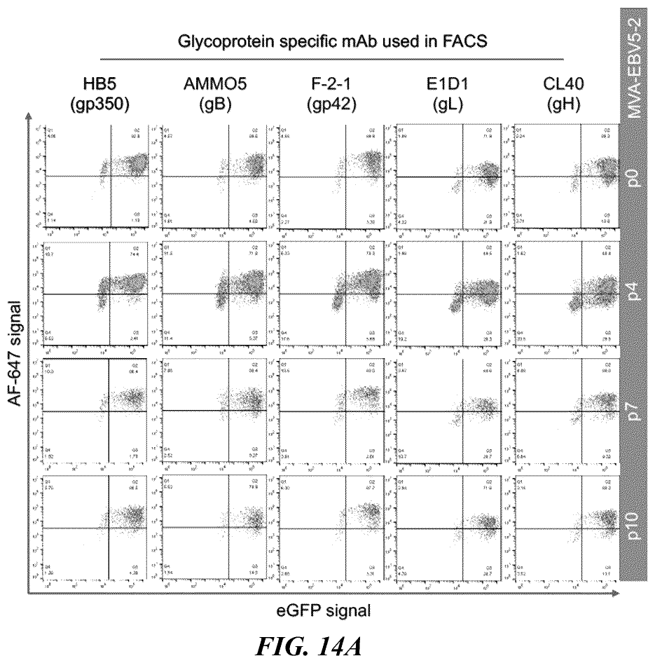

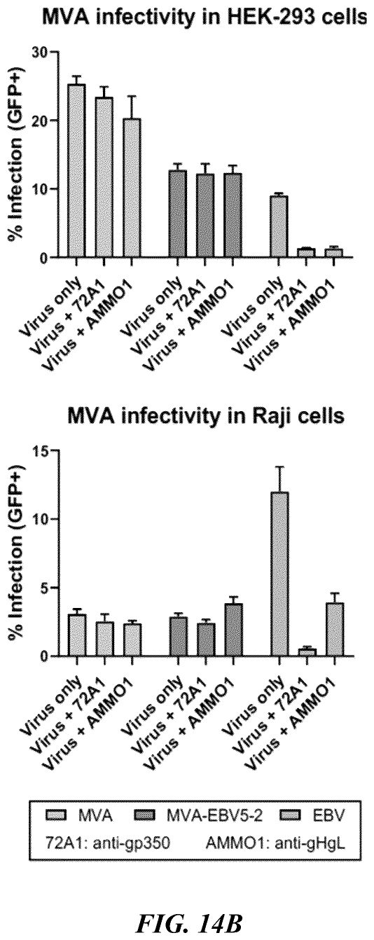

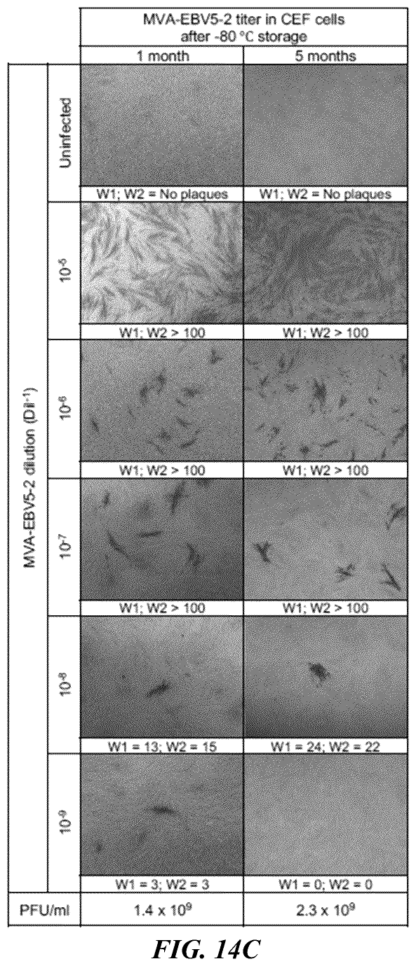

As stated above, the inserted glycoprotein expression cassettes in MVA-EBV5-2 were assessed to determine whether translationally active and stable after repeated viral passaging, an important quality for large-scale manufacturing of viral vector vaccines (FIGS. 1B-1E). Representative flow cytometry plots for data presented in FIG. 1E are shown in FIG. 14A. Taken together, these results demonstrate that MVA-EBV5-2 is stable, genetically and translationally, through ten viral passages.

Because MVA-EBV5-2 incorporates EBV entry glycoproteins, the presence of these glycoproteins was tested to determine if they could provide MVA with an alternative infection pathway into EBV-susceptible human cells. To do this, the EBV-susceptible human cell lines HEK-293 and Raji with MVA-EBV5-2 or wildtype MVA, was tested in the presence or absence of the EBV-specific neutralizing antibodies 72A1 (anti-gp350,²³) and AMMO1 (anti-gHgL,²⁸) (FIG. 14B). In both cell lines, the neutralizing antibodies efficiently reduced infection of EBV, which was used as a control; however, while both MVA viruses were able to infect each cell line, the antibodies did not reduce infection of either virus, suggesting that the expression of EBV entry glycoproteins did not affect the tropism of MVA towards these cells. As a final characterization step, the titer stability of an MVA-EBV5-2 batch after 1 or 5 months in −80° C. storage was tested following production, using a PFU immunostaining assay, currently considered one of the traditional methods for MVA titration^{101, 107} (FIG. 14C). Results of the assay demonstrated that MVA-EBV5-2 titers are maintained in up to 5 months in this storage condition, assuring its stability during immunization protocols. The immunogenicity of MVA-EBV5-2 is characterized in Examples 2-4 above.

In summary, the present technology demonstrates that a multivalent MVA-based vaccine targeting multiple EBV entry glycoproteins is immunogenic in mice and rhesus macaques and provides superior protection to NSG huMice against EBV challenge when compared to a monovalent gp350-based vaccine.

From the foregoing, it will be appreciated that specific embodiments of the invention have been described herein for purposes of illustration, but that various modifications may be made without deviating from the scope of the invention. Accordingly, the invention is not limited except as by the appended claims.

### REFERENCES

ADDIN EN.REFLIST 1. Cohen, J. I., Fauci, A. S., Varmus, H., and Nabel, G. J. (2011). Epstein-Barr virus: an important vaccine target for cancer prevention. Sci Transl Med 3, 107fs107. 10.1126/scitranslmed.3002878.
2. Wong, Y., Meehan, M. T., Burrows, S. R., Doolan, D. L., and Miles, J. J. (2022). Estimating the global burden of Epstein-Barr virus-related cancers. J Cancer Res Clin Oncol 148, 31-46. 10.1007/s00432-021-03824-y.
3. Bjornevik, K., Cortese, M., Healy, B. C., Kuhle, J., Mina, M. J., Leng, Y., Elledge, S. J., Niebuhr, D. W., Scher, A. I., Munger, K. L., and Ascherio, A. (2022). Longitudinal analysis reveals high prevalence of Epstein-Barr virus associated with multiple sclerosis. Science 375, 296-301. 10.1126/science.abj8222.
4. Houen, G., and Trier, N. H. (2020). Epstein-Barr Virus and Systemic Autoimmune Diseases. Front Immunol 11, 587380. 10.3389/fimmu.2020.587380.
5. Lanz, T. V., Brewer, R. C., Ho, P. P., Moon, J. S., Jude, K. M., Fernandez, D., Fernandes, R. A., Gomez, A. M., Nadj, G. S., Bartley, C. M., et al. (2022). Clonally expanded B cells in multiple sclerosis bind EBV EBNA1 and GlialCAM. Nature. 10.1038/s41586-022-04432-7.
6. Escalante, G. M., Mutsvunguma, L. Z., Muniraju, M., Rodriguez, E., and Ogembo, J. G. (2022). Four Decades of Prophylactic EBV Vaccine Research: A Systematic Review and Historical Perspective. Front Immunol 13, 867918. 10.3389/fimmu.2022.867918.
7. Connolly, S. A., Jackson, J. O., Jardetzky, T. S., and Longnecker, R. (2011). Fusing structure and function: a structural view of the herpesvirus entry machinery. Nat Rev Microbiol 9, 369-381. 10.1038/nrmicro2548.
8. Chen, J., Sathiyamoorthy, K., Zhang, X., Schaller, S., Perez White, B. E., Jardetzky, T. S., and Longnecker, R. (2018). Ephrin receptor A2 is a functional entry receptor for Epstein-Barr virus. Nature microbiology 3, 172-180. 10.1038/s41564-017-0081-7.
9. Zhang, H., Li, Y., Wang, H. B., Zhang, A., Chen, M. L., Fang, Z. X., Dong, X. D., Li, S. B., Du, Y., Xiong, D., et al. (2018). Ephrin receptor A2 is an epithelial cell receptor for Epstein-Barr virus entry. Nature microbiology 3, 1-8. 10.1038/s41564-017-0080-8.
10. Xiong, D., Du, Y., Wang, H. B., Zhao, B., Zhang, H., Li, Y., Hu, L. J., Cao, J. Y., Zhong, Q., Liu, W.L., et al. (2015). Nonmuscle myosin heavy chain IIA mediates Epstein-Barr virus infection of nasopharyngeal epithelial cells. Proceedings of the National Academy of Sciences of the United States of America 112, 11036-11041. 10.1073/pnas. 1513359112.
11. Wang, H. B., Zhang, H., Zhang, J. P., Li, Y., Zhao, B., Feng, G. K., Du, Y., Xiong, D., Zhong, Q., Liu, W. L., et al. (2015). Neuropilin 1 is an entry factor that promotes EBV infection of nasopharyngeal epithelial cells. Nat Commun 6, 6240. 10.1038/ncomms7240.
12. Ogembo, J. G., Kannan, L., Ghiran, I., Nicholson-Weller, A., Finberg, R. W., Tsokos, G. C., and Fingeroth, J. D. (2013). Human complement receptor type 1/CD35 is an Epstein-Barr Virus receptor. Cell Rep 3, 371-385. 10.1016/j.celrep.2013.01.023.
13. Fingeroth, J. D., Weis, J. J., Tedder, T. F., Strominger, J. L., Biro, P. A., and Fearon, D. T. (1984). Epstein-Barr virus receptor of human B lymphocytes is the C3d receptor CR2. Proceedings of the National Academy of Sciences of the United States of America 81, 4510-4514. 10.1073/pnas.81.14.4510.
14. Tanner, J., Weis, J., Fearon, D., Whang, Y., and Kieff, E. (1987). Epstein-Barr virus gp350/220 binding to the B lymphocyte C3d receptor mediates adsorption, capping, and endocytosis. Cell 50, 203-213.
15. Spriggs, M. K., Armitage, R. J., Comeau, M. R., Strockbine, L., Farrah, T., Macduff, B., Ulrich, D., Alderson, M. R., Müllberg, J., and Cohen, J. I. (1996). The extracellular domain of the Epstein-Barr virus BZLF2 protein binds the HLA-DR beta chain and inhibits antigen presentation. Journal of virology 70, 5557-5563. doi: 10.1128/jvi.70.8.5557-5563.1996.
16. Li, Q., Spriggs, M. K., Kovats, S., Turk, S. M., Comeau, M. R., Nepom, B., and Hutt-Fletcher, L. M. (1997). Epstein-Barr virus uses HLA class Il as a cofactor for infection of B lymphocytes. Journal of virology 71, 4657-4662.
17. Haan, K. M., Kwok, W. W., Longnecker, R., and Speck, P. (2000). Epstein-Barr virus entry utilizing HLA-DP or HLA-DQ as a coreceptor. Journal of virology 74, 2451-2454. 10.1128/jvi.74.5.2451-2454.2000.
18. Gu, S. Y., Huang, T. M., Ruan, L., Miao, Y. H., Lu, H., Chu, C. M., Motz, M., and Wolf, H. (1995). First EBV vaccine trial in humans using recombinant vaccinia virus expressing the major membrane antigen. Developments in biological standardization 84, 171-177.
19 Sokal, E. M., Hoppenbrouwers, K., Vandermeulen, C., Moutschen, M., Leonard, P., Moreels, A., Haumont, M., Bollen, A., Smets, F., and Denis, M. (2007). Recombinant gp350 vaccine for infectious mononucleosis: a phase 2, randomized, double-blind, placebo-controlled trial to evaluate the safety, immunogenicity, and efficacy of an Epstein-Barr virus vaccine in healthy young adults. J Infect Dis 196, 1749-1753. 10.1086/523813.
20. Moutschen, M., Leonard, P., Sokal, E. M., Smets, F., Haumont, M., Mazzu, P., Bollen, A., Denamur, F., Peeters, P., Dubin, G., and Denis, M. (2007). Phase I/II studies to evaluate safety and immunogenicity of a recombinant gp350 Epstein-Barr virus vaccine in healthy adults. Vaccine 25, 4697-4705. 10.1016/j.vaccine.2007.04.008.
21. Rees, L., Tizard, E. J., Morgan, A. J., Cubitt, W. D., Finerty, S., Oyewole-Eletu, T. A., Owen, K., Royed, C., Stevens, S. J., Shroff, R. C., et al. (2009). A phase I trial of epstein-barr virus gp350 vaccine for children with chronic kidney disease awaiting transplantation. Transplantation 88, 1025-1029. 10.1097/TP.0b013e3181b9d918.
22. Janz, A., Oezel, M., Kurzeder, C., Mautner, J., Pich, D., Kost, M., Hammerschmidt, W., and Delecluse, H.J. (2000). Infectious Epstein-Barr virus lacking major glycoprotein BLLF1 (gp350/220) demonstrates the existence of additional viral ligands. Journal of virology 74, 10142-10152. 10.1128/jvi.74.21.10142-10152.2000.
23. Hoffman, G. J., Lazarowitz, S. G., and Hayward, S. D. (1980). Monoclonal antibody against a 250,000-dalton glycoprotein of Epstein-Barr virus identifies a membrane antigen and a neutralizing antigen. Proceedings of the National Academy of Sciences of the United States of America 77, 2979-2983. 10.1073/pnas.77.5.2979.
24. Thorley-Lawson, D. A., and Geilinger, K. (1980). Monoclonal antibodies against the major glycoprotein (gp350/220) of Epstein-Barr virus neutralize infectivity. Proceedings of the National Academy of Sciences of the United States of America 77, 5307-5311. 10.1073/pnas.77.9.5307.
25. Strnad, B. C., Schuster, T., Klein, R., Hopkins, R. F., 3rd, Witmer, T., Neubauer, R. H., and Rabin, H. (1982). Production and characterization of monoclonal antibodies against the Epstein-Barr virus membrane antigen. Journal of virology 41, 258-264. 10.1128/JVI.41.1.258-264.1982.
26. Oba, D. E., and Hutt-Fletcher, L. M. (1988). Induction of antibodies to the Epstein-Barr virus glycoprotein gp85 with a synthetic peptide corresponding to a sequence in the BXLF2 open reading frame. Journal of virology 62, 1108-1114. 10.1128/JVI.62.4.1108-1114.1988.
27. Molesworth, S. J., Lake, C. M., Borza, C. M., Turk, S. M., and Hutt-Fletcher, L. M. (2000). Epstein-Barr virus gH is essential for penetration of B cells but also plays a role in attachment of virus to epithelial cells. Journal of virology 74, 6324-6332. 10.1128/jvi.74.14.6324-6332.2000.
28. Snijder, J., Ortego, M. S., Weidle, C., Stuart, A. B., Gray, M. D., McElrath, M. J., Pancera, M., Veesler, D., and McGuire, A. T. (2018). An Antibody Targeting the Fusion Machinery Neutralizes Dual-Tropic Infection and Defines a Site of Vulnerability on Epstein-Barr Virus. Immunity 48, 799-811 e799. 10.1016/j.immuni.2018.03.026.
29. Bu, W., Joyce, M. G., Nguyen, H., Banh, D. V., Aguilar, F., Tariq, Z., Yap, M. L., Tsujimura, Y., Gillespie, R. A., Tsybovsky, Y., et al. (2019). Immunization with Components of the Viral Fusion Apparatus Elicits Antibodies That Neutralize Epstein-Barr Virus in B Cells and Epithelial Cells. Immunity 50, 1305-1316 e1306. 10.1016/j.immuni.2019.03.010.
30. Mutsvunguma, L. Z., Rodriguez, E., Escalante, G. M., Muniraju, M., Williams, J. C., Warden, C., Qin, H., Wang, J., Wu, X., Barasa, A., et al. (2019). Identification of multiple potent neutralizing and non-neutralizing antibodies against Epstein-Barr virus gp350 protein with potential for clinical application and as reagents for mapping immunodominant epitopes. Virology 536, 1-15. 10.1016/j.virol.2019.07.026.
31. Zhu, Q. Y., Shan, S., Yu, J., Peng, S. Y., Sun, C., Zuo, Y., Zhong, L. Y., Yan, S. M., Zhang, X., Yang, Z., et al. (2021). A potent and protective human neutralizing antibody targeting a novel vulnerable site of Epstein-Barr virus. Nat Commun 12, 6624. 10.1038/s41467-021-26912-6.
32. Hong, J., Wei, D., Zhong, L., Wu, Q., Chen, K., Zhang, W., Yang, Y., Chen, J., Xia, N., Zhang, X., and Chen, Y. (2022). Glycoprotein B Antibodies Completely Neutralize EBV Infection of B Cells. Front Immunol 13, 920467. 10.3389/fimmu.2022.920467.
33. Hong, J., Zhong, L., Zheng, Q., Wu, Q., Zha, Z., Wei, D., Chen, H., Zhang, W., Zhang, S., Huang, Y., et al. (2022). A Neutralizing Antibody Targeting gH Provides Potent Protection against EBV Challenge In Vivo. Journal of virology 96, e0007522. 10.1128/jvi.00075-22.
34. Zhang, X., Hong, J., Zhong, L., Wu, Q., Zhang, S., Zhu, Q., Chen, H., Wei, D., Li, R., Zhang, W., et al. (2022). Protective anti-gB neutralizing antibodies targeting two vulnerable sites for EBV-cell membrane fusion. Proceedings of the National Academy of Sciences of the United States of America 119, e2202371119. 10.1073/pnas.2202371119.
35. Chen, W. H., Kim, J., Bu, W., Board, N. L., Tsybovsky, Y., Wang, Y., Hostal, A., Andrews, S. F., Gillespie, R. A., Choe, M., et al. (2022). Epstein-Barr virus gH/gL has multiple sites of vulnerability for virus neutralization and fusion inhibition. Immunity 55, 2135-2148.e2136. 10.1016/j.immuni.2022.10.003.
36. Taylor, G. S., Long, H. M., Brooks, J. M., Rickinson, A. B., and Hislop, A. D. (2015). The Immunology of Epstein-Barr Virus-Induced Disease. Annual review of immunology 33, 787-821. 10.1146/annurev-immunol-032414-112326.
37. Long, H. M., Leese, A. M., Chagoury, O. L., Connerty, S. R., Quarcoopome, J., Quinn, L.L., Shannon-Lowe, C., and Rickinson, A.B. (2011). Cytotoxic CD4+ T cell responses to EBV contrast with CD8 responses in breadth of lytic cycle antigen choice and in lytic cycle recognition. J Immunol 187, 92-101. 10.4049/jimmunol. 1100590.
38. Adhikary, D., Behrends, U., Moosmann, A., Witter, K., Bornkamm, G. W., and Mautner, J. (2006). Control of Epstein-Barr virus infection in vitro by T helper cells specific for virion glycoproteins. J Exp Med 203, 995-1006. 10.1084/jem.20051287.
39. Wallace, L. E., Wright, J., Ulaeto, D. O., Morgan, A. J., and Rickinson, A. B. (1991). Identification of two T-cell epitopes on the candidate Epstein-Barr virus vaccine glycoprotein gp340 recognized by CD4+ T-cell clones. Journal of virology 65, 3821-3828. 10.1128/jvi.65.7.3821-3828.1991.
40. Khanna, R., Sherritt, M., and Burrows, S. R. (1999). EBV structural antigens, gp350 and gp85, as targets for ex vivo virus-specific CTL during acute infectious mononucleosis: potential use of gp350/gp85 CTL epitopes for vaccine design. J Immunol 162, 3063-3069.
41. Abbott, R. J., Quinn, L. L., Leese, A. M., Scholes, H. M., Pachnio, A., and Rickinson, A.B. (2013). CD8+T cell responses to lytic EBV infection: late antigen specificities as subdominant components of the total response. J Immunol 191, 5398-5409. 10.4049/jimmunol. 1301629.
42. Pudney, V. A., Leese, A. M., Rickinson, A. B., and Hislop, A. D. (2005). CD8+ immunodominance among Epstein-Barr virus lytic cycle antigens directly reflects the efficiency of antigen presentation in lytically infected cells. J Exp Med 201, 349-360. 10.1084/jem.20041542.
43. Khanna, R., and Burrows, S. R. (2000). Role of cytotoxic T lymphocytes in Epstein-Barr virus-associated diseases. Annu Rev Microbiol 54, 19-48. 10.1146/annurev.micro.54.1.19.
44. Chen, H., Zhang, X., Zhang, S., Duan, X., Xiang, T., Zhou, X., Zhang, W., Zhang, X., Feng, Q., Kang, Y., et al. (2021). T cell epitope screening of Epstein-Barr virus fusion protein gB. Journal of virology 95. 10.1128/jvi.00081-21.
45. Wei, C. J., Bu, W., Nguyen, L. A., Batchelor, J. D., Kim, J., Pittaluga, S., Fuller, J. R., Nguyen, H., Chou, T. H., Cohen, J. I., and Nabel, G. J. (2022). A bivalent Epstein-Barr virus vaccine induces neutralizing antibodies that block infection and confer immunity in humanized mice. Sci Transl Med 14, eabf3685. 10.1126/scitranslmed.abf3685.
46. Cui, X., Cao, Z., Ishikawa, Y., Cui, S., Imadome, K. I., and Snapper, C. M. (2021). Immunization with Epstein-Barr Virus Core Fusion Machinery Envelope Proteins Elicit High Titers of Neutralizing Activities and Protect Humanized Mice from Lethal Dose EBV Challenge. Vaccines (Basel) 9. 10.3390/vaccines9030285.
47. Perez, E. M., Foley, J., Tison, T., Silva, R., and Ogembo, J. G. (2017). Novel Epstein-Barr virus-like particles incorporating gH/gL-EBNA1 or gB-LMP2 induce high neutralizing antibody titers and EBV-specific T-cell responses in immunized mice. Oncotarget 8, 19255-19273. 10.18632/oncotarget. 13770.
48. Escalante, G.M., Foley, J., Mutsvunguma, L.Z., Rodriguez, E., Mulama, D.H., Muniraju, M., Ye, P., Barasa, A.K., and Ogembo, J.G. (2020). A Pentavalent Epstein-Barr Virus-Like Particle Vaccine Elicits High Titers of Neutralizing Antibodies against Epstein-Barr Virus Infection in Immunized Rabbits. Vaccines (Basel) 8, 169. 10.3390/vaccines8020169.
49. Ura, T., Okuda, K., and Shimada, M. (2014). Developments in Viral Vector-Based Vaccines. Vaccines (Basel) 2, 624-641. 10.3390/vaccines2030624.
50. Hofmeyer, K. A., Bianchi, K. M., and Wolfe, D. N. (2022). Utilization of Viral Vector Vaccines in Preparing for Future Pandemics. Vaccines 10, 436.
51. Mayr, A., Stickl, H., Müller, H. K., Danner, K., and Singer, H. (1978). [The smallpox vaccination strain MVA: marker, genetic structure, experience gained with the parenteral vaccination and behavior in organisms with a debilitated defence mechanism (author's transl)]. Zentralbl Bakteriol B 167, 375-390.
52. Orlova, O. V., Glazkova, D. V., Bogoslovskaya, E. V., Shipulin, G. A., and Yudin, S. M. (2022). Development of Modified Vaccinia Virus Ankara-Based Vaccines: Advantages and Applications. Vaccines (Basel) 10. 10.3390/vaccines10091516.
53. Cottingham, M. G., and Carroll, M. W. (2013). Recombinant MVA vaccines: dispelling the myths. Vaccine 31, 4247-4251.
54. Gómez Carmen, E., Nájera José, L., Perdiguero, B., García-Arriaza, J., Sorzano Carlos Oscar, S., Jiménez, V., González-Sanz, R., Jiménez José, L., Muñoz-Fernández María, A., López Bernaldo de Quirós Juan, C., et al. (2011). The HIV/AIDS Vaccine Candidate MVA-B Administered as a Single Immunogen in Humans Triggers Robust, Polyfunctional, and Selective Effector Memory T Cell Responses to HIV-1 Antigens. Journal of virology 85, 11468-11478. 10.1128/JVI.05165-11.
55. Antrobus, R. D., Lillie, P. J., Berthoud, T. K., Spencer, A. J., McLaren, J. E., Ladell, K., Lambe, T., Milicic, A., Price, D. A., Hill, A. V. S., and Gilbert, S. C. (2012). A T Cell-Inducing Influenza Vaccine for the Elderly: Safety and Immunogenicity of MVA-NP+M1 in Adults Aged over 50 Years. PLOS ONE 7, e48322. 10.1371/journal.pone.0048322.
56. La Rosa, C., Longmate, J., Martinez, J., Zhou, Q., Kaltcheva, T. I., Tsai, W., Drake, J., Carroll, M., Wussow, F., Chiuppesi, F., et al. (2017). MVA vaccine encoding CMV antigens safely induces durable expansion of CMV-specific T cells in healthy adults. Blood 129, 114-125. 10.1182/blood-2016-07-729756.
57. Domi, A., Feldmann, F., Basu, R., McCurley, N., Shifflett, K., Emanuel, J., Hellerstein, M. S., Guirakhoo, F., Orlandi, C., Flinko, R., et al. (2018). A Single Dose of Modified Vaccinia Ankara expressing Ebola Virus Like Particles Protects Nonhuman Primates from Lethal Ebola Virus Challenge. Sci Rep 8, 864. 10.1038/s41598-017-19041-y.
58. Pittman, P. R., Hahn, M., Lee, H. S., Koca, C., Samy, N., Schmidt, D., Hornung, J., Weidenthaler, H., Heery, C. R., Meyer, T. P. H., et al. (2019). Phase 3 Efficacy Trial of Modified Vaccinia Ankara as a Vaccine against Smallpox. N Engl J Med 381, 1897-1908. 10.1056/NEJMoa1817307.
59. Jordan, E., Lawrence, S. J., Meyer, T. P. H., Schmidt, D., Schultz, S., Mueller, J., Stroukova, D., Koenen, B., Gruenert, R., Silbernagl, G., et al. (2021). Broad Antibody and Cellular Immune Response From a Phase 2 Clinical Trial With a Novel Multivalent Poxvirus-Based Respiratory Syncytial Virus Vaccine. The Journal of Infectious Diseases 223, 1062-1072. 10.1093/infdis/jiaa460.
60. Chiuppesi, F., Zaia, J. A., Frankel, P. H., Stan, R., Drake, J., Williams, B., Acosta, A. M., Francis, K., Taplitz, R. A., Dickter, J. K., et al. (2022). Safety and immunogenicity of a synthetic multiantigen modified vaccinia virus Ankara-based COVID-19 vaccine (COH04S1): an open-label and randomised, phase 1 trial. Lancet Microbe 3, e252-e264. 10.1016/s2666-5247 (22) 00027-1.
61. Wussow, F., Chiuppesi, F., Meng, Z., Martinez, J., Nguyen, J., Barry, P. A., and Diamond, D. J. (2018). Exploiting 2A peptides to elicit potent neutralizing antibodies by a multi-subunit herpesvirus glycoprotein complex. J Virol Methods 251, 30-37. 10.1016/j.jviromet.2017.10.006.
62. Tischer, B. K., Smith, G. A., and Osterrieder, N. (2010). En passant mutagenesis: a two step markerless red recombination system. Methods Mol Biol 634, 421-430. 10.1007/978-1-60761-652-8_30.
63. Wang, Z., Martinez, J., Zhou, W., La Rosa, C., Srivastava, T., Dasgupta, A., Rawal, R., Li, Z., Britt, W. J., and Diamond, D. (2010). Modified H5 promoter improves stability of insert genes while maintaining immunogenicity during extended passage of genetically engineered MVA vaccines. Vaccine 28, 1547-1557.
64. Kim, J. H., Lee, S. R., Li, L. H., Park, H. J., Park, J. H., Lee, K. Y., Kim, M. K., Shin, B. A., and Choi, S. Y. (2011). High cleavage efficiency of a 2A peptide derived from porcine teschovirus-1 in human cell lines, zebrafish and mice. PLOS One 6, e18556. 10.1371/journal.pone.0018556.
65. Kanekiyo, M., Bu, W., Joyce, M. G., Meng, G., Whittle, J. R. R., Baxa, U., Yamamoto, T., Narpala, S., Todd, J.-P., Rao, S.S., et al. (2015). Rational Design of an Epstein-Barr Virus Vaccine Targeting the Receptor-Binding Site. Cell 162, 1090-1100. 10.1016/j.cell.2015.07.043.
66. Kang, Y. F., Zhang, X., Yu, X. H., Zheng, Q., Liu, Z., Li, J. P., Sun, C., Kong, X. W., Zhu, Q. Y., Chen, H. W., et al. (2021). Immunization with a Self-Assembled Nanoparticle Vaccine Elicits Potent Neutralizing Antibody Responses against EBV Infection. Nano Lett 21, 2476-2486. 10.1021/acs.nanolett.0c04687.
67. Chiuppesi, F., Nguyen, J., Park, S., Contreras, H., Kha, M., Meng, Z., Kaltcheva, T., Iniguez, A., Martinez, J., La Rosa, C., et al. (2018). Multiantigenic Modified Vaccinia Virus Ankara Vaccine Vectors To Elicit Potent Humoral and Cellular Immune Reponses against Human Cytomegalovirus in Mice. Journal of virology 92. 10.1128/jvi.01012-18.
68. Frey, S. E., Stapleton, J. T., Ballas, Z. K., Rasmussen, W. L., Kaufman, T. M., Blevins, T. P., Jensen, T. L., Davies, D. H., Tary-Lehmann, M., Chaplin, P., et al. (2021). Human Antibody Responses Following Vaccinia Immunization Using Protein Microarrays and Correlation With Cell-Mediated Immunity and Antibody-Dependent Cellular Cytotoxicity Responses. J Infect Dis 224, 1372-1382. 10.1093/infdis/jiab111.
69. Carville, A., and Mansfield, K. G. (2008). Comparative pathobiology of macaque lymphocryptoviruses. Comp Med 58, 57-67.
70. Sonntag, K., Eckert, F., Welker, C., Müller, H., Müller, F., Zips, D., Sipos, B., Klein, R., Blank, G., Feuchtinger, T., et al. (2015). Chronic graft-versus-host-disease in CD34+-humanized NSG mice is associated with human susceptibility HLA haplotypes for autoimmune disease. Journal of Autoimmunity 62, 55-66.
71. Snapper, C. M., and Paul, W. E. (1987). Interferon-gamma and B cell stimulatory factor-1 reciprocally regulate Ig isotype production. Science 236, 944-947. 10.1126/science.3107127.
72. Chaplin, D. D. (2010). Overview of the immune response. J Allergy Clin Immunol 125, S3-23. 10.1016/j.jaci.2009.12.980.
73. Marcotte, H., and Lavoie, M. C. (1998). Oral microbial ecology and the role of salivary immunoglobulin A. Microbiol Mol Biol Rev 62, 71-109. 10.1128/mmbr.62.1.71-109.1998.
74. Americo, J. L., Cotter, C. A., Earl, P. L., Liu, R., and Moss, B. (2022). Intranasal inoculation of an MVA-based vaccine induces IgA and protects the respiratory tract of hACE2 mice from SARS-COV-2 infection. Proceedings of the National Academy of Sciences 119, e2202069119. 10.1073/pnas.2202069119.
75. Pierantoni, A., Esposito, M. L., Ammendola, V., Napolitano, F., Grazioli, F., Abbate, A., Del Sorbo, M., Siani, L., D′Alise, A.M., Taglioni, A., et al. (2015). Mucosal delivery of a vectored RSV vaccine is safe and elicits protective immunity in rodents and nonhuman primates. Mol Ther Methods Clin Dev 2, 15018. 10.1038/mtm.2015.18.
76. Brandtzaeg, P. (2007). Do salivary antibodies reliably reflect both mucosal and systemic immunity? Ann N Y Acad Sci 1098, 288-311. 10.1196/annals. 1384.012.
77. Kadelka, C., Liechti, T., Ebner, H., Schanz, M., Rusert, P., Friedrich, N., Stiegeler, E., Braun, D. L., Huber, M., Scherrer, A. U., et al. (2018). Distinct, IgG1-driven antibody response landscapes demarcate individuals with broadly HIV-1 neutralizing activity. Journal of Experimental Medicine 215, 1589-1608. 10.1084/jem.20180246.
78. Hofmeister, Y., Planitzer, C. B., Farcet, M. R., Teschner, W., Butterweck, H. A., Weber, A., Holzer, G. W., and Kreil, T. R. (2011). Human IgG subclasses: in vitro neutralization of and in vivo protection against West Nile virus. Journal of virology 85, 1896-1899. 10.1128/jvi.02155-10.
79. Huber, V. C., Mckeon, R. M., Brackin, M. N., Miller, L. A., Keating, R., Brown, S. A., Makarova, N., Perez, D. R., Macdonald, G. H., and McCullers, J. A. (2006). Distinct contributions of vaccine-induced immunoglobulin G1 (IgG1) and IgG2a antibodies to protective immunity against influenza. Clin Vaccine Immunol 13, 981-990. 10.1128/cvi.00156-06.
80. Chen, W., Zhang, L., Li, J., Bai, S., Wang, Y., Zhang, B., Zheng, Q., Chen, M., Zhao, W., and Wu, J. (2022). The kinetics of IgG subclasses and contributions to neutralizing activity against SARS-COV-2 wild-type strain and variants in healthy adults immunized with inactivated vaccine. Immunology 167, 221-232.
81. Mathiesen, T., Persson, M. A., Sundqvist, V. A., and Wahren, B. (1988). Neutralization capacity and antibody dependent cell-mediated cytotoxicity of separated IgG subclasses 1, 3 and 4 against herpes simplex virus. Clin Exp Immunol 72, 211-215.
82. Malhi, H., Homad, L. J., Wan, Y. H., Poudel, B., Fiala, B., Borst, A. J., Wang, J. Y., Walkey, C., Price, J., Wall, A., et al. (2022). Immunization with a self-assembling nanoparticle vaccine displaying EBV gH/gL protects humanized mice against lethal viral challenge. Cell Rep Med 3, 100658. 10.1016/j.xcrm.2022.100658.
83. Kim, J., Bu, W., Mine, S., Tariq, Z., Nguyen, H., Wang, Y., Tolman, C., Mond, J., and Cohen, J. I. (2021). Epstein-Barr virus (EBV) hyperimmune globulin isolated from donors with high gp350 antibody titers protect humanized mice from challenge with EBV. Virology 561, 80-86. 10.1016/j.virol.2021.06.006.
84. Singh, S., Homad, L. J., Akins, N. R., Stoffers, C. M., Lackhar, S., Malhi, H., Wan, Y. H., Rawlings, D. J., and McGuire, A. T. (2020). Neutralizing Antibodies Protect against Oral Transmission of Lymphocryptovirus. Cell Rep Med 1. 10.1016/j.xcrm.2020.100033.
85. Haque, T., Johannessen, I., Dombagoda, D., Sengupta, C., Burns, D. M., Bird, P., Hale, G., Mieli-Vergani, G., and Crawford, D. H. (2006). A mouse monoclonal antibody against Epstein-Barr virus envelope glycoprotein 350 prevents infection both in vitro and in vivo. J Infect Dis 194, 584-587. 10.1086/505912.
86. Ishikawa, F., Yasukawa, M., Lyons, B., Yoshida, S., Miyamoto, T., Yoshimoto, G., Watanabe, T., Akashi, K., Shultz, L. D., and Harada, M. (2005). Development of functional human blood and immune systems in NOD/SCID/IL2 receptor y chainnull mice. Blood 106, 1565-1573.
87. Fujiwara, S., Matsuda, G., and Imadome, K. (2013). Humanized mouse models of epstein-barr virus infection and associated diseases. Pathogens 2, 153-176. 10.3390/pathogens2010153.
88. Münz, C. (2017). Humanized mouse models for Epstein Barr virus infection. Current Opinion in Virology 25, 113-118.
89. Cox, C., Naylor, B. A., Mackett, M., Arrand, J. R., Griffin, B. E., and Wedderburn, N. (1998). Immunization of common marmosets with Epstein-Barr virus (EBV) envelope glycoprotein gp340: effect on viral shedding following EBV challenge. J Med Virol 55, 255-261. 10.1002/(sici) 1096-9071 (199808)55:4<255::aid-jmv1>3.0.co;2=#.
90. Mackett, M., Cox, C., Pepper, S. D., Lees, J. F., Naylor, B. A., Wedderburn, N., and Arrand, J. R. (1996). Immunisation of common marmosets with vaccinia virus expressing Epstein-Barr virus (EBV) gp340 and challenge with EBV. J Med Virol 50, 263-271. 10.1002/(SICI) 1096-9071 (199611)50:3<263::AID-JMV9>3.0.CO;2-7.
91. Wang, F. (2013). Nonhuman primate models for Epstein-Barr virus infection. Curr Opin Virol 3, 233-237. 10.1016/j.coviro.2013.03.003.
92. ClinicalTrials.gov [Internet] (2020). Safety and Immunogenicity of an Epstein-Barr Virus (EBV) gp350-Ferritin Nanoparticle Vaccine in Healthy Adults With or Without EBV Infection. Identifier NCT04645147.
93. ClinicalTrials.gov [Internet] (2023). Trial Evaluating the Immunogenicity and Safety of an Adjuvanted Epstein-Barr Virus (EBV) Glycoprotein 350 Vaccine in EBV-seronegative Persons. Identifier NCT05683834.
94. ClinicalTrials.gov [Internet] (2021). A Study of an Epstein-Barr Virus (EBV) Candidate Vaccine, mRNA-1189, in 18-to 30-Year-Old Healthy Adults. Identifier NCT05164094.
95. Schmelz, M., Sodeik, B., Ericsson, M., Wolffe, E. J., Shida, H., Hiller, G., and Griffiths, G. (1994). Assembly of vaccinia virus: the second wrapping cisterna is derived from the trans Golgi network. Journal of virology 68, 130-147. doi: 10.1128/jvi.68.1.130-147.1994.
96. Kimura, H., Morita, M., Yabuta, Y., Kuzushima, K., Kato, K., Kojima, S., Matsuyama, T., and Morishima, T. (1999). Quantitative analysis of Epstein-Barr virus load by using a real-time PCR assay. J Clin Microbiol 37, 132-136. 10.1128/jcm.37.1.132-136.1999.
97. Abramson, J. H. (2011). WINPEPI updated: computer programs for epidemiologists, and their teaching potential. Epidemiologic Perspectives & Innovations 8, 1. 10.1186/1742-5573 Aug. 1.
98. Niwa, H., Yamamura, K., and Miyazaki, J. (1991). Efficient selection for high-expression transfectants with a novel eukaryotic vector. Gene 108, 193-199. 10.1016/0378-1119 (91) 90434-d.
99. Chapple, S. D. J., Crofts, A. M., Shadbolt, S. P., McCafferty, J., and Dyson, M. R. (2006). Multiplexed expression and screening for recombinant protein production in mammalian cells. BMC Biotechnology 6, 49. 10.1186/1472-6750-6-49.
100. Durocher, Y., Perret, S., and Kamen, A. (2002). High-level and high-throughput recombinant protein production by transient transfection of suspension-growing human 293-EBNA1 cells. Nucleic Acids Research 30, e9-e9. 10.1093/nar/30.2.e9.
101. Cotter, C. A., Earl, P. L., Wyatt, L. S., and Moss, B. (2015). Preparation of Cell Cultures and Vaccinia Virus Stocks. Curr Protoc Microbiol 39, 14a. 13.11-14a. 13.18. 10.1002/9780471729259.mc14a03s39.
102. Kremer, M., Volz, A., Kreijtz, J. H. C. M., Fux, R., Lehmann, M. H., and Sutter, G. (2012). Easy and Efficient Protocols for Working with Recombinant Vaccinia Virus MVA. In Vaccinia Virus and Poxvirology: Methods and Protocols, S. N. Isaacs, ed. (Humana Press), pp. 59-92. 10.1007/978-1-61779-876-4_4.
103. Li, Z., Ling, L., Liu, X., Laus, R., and Delcayre, A. (2010). A flow cytometry-based immuno-titration assay for rapid and accurate titer determination of modified vaccinia Ankara virus vectors. J Virol Methods 169, 87-94. 10.1016/j.jviromet.2010.07.003.
104. Schmelz, M., Sodeik, B., Ericsson, M., Wolffe, E. J., Shida, H., Hiller, G., and Griffiths, G. (1994). Assembly of vaccinia virus: the second wrapping cisterna is derived from the trans Golgi network. Journal of virology 68, 130-147. 10.1128/jvi.68.1.130-147.1994.
105. Volk, V., Theobald, S. J., Danisch, S., Khailaie, S., Kalbarczyk, M., Schneider, A., Bialek-Waldmann, J., Krönke, N., Deng, Y., Eiz-Vesper, B., et al. (2021). PD-1 Blockade Aggravates Epstein-Barr Virus+Post-Transplant Lymphoproliferative Disorder in Humanized Mice Resulting in Central Nervous System Involvement and CD4+ T Cell Dysregulations. Frontiers in oncology 10. 10.3389/fonc.2020.614876.
106. Liu Z, Chen O, Wall JBJ, Zheng M, Zhou Y, Wang L, et al. Systematic comparison of 2A peptides for cloning multi-genes in a polycistronic vector. Sci Rep. (2017) 7:2193. doi: 10.1038/s41598-017-02460-2.
107. Staib C, Drexler I, Sutter G. Construction and isolation of recombinant MVA. Methods Mol Biol. (2004) 269:77-100. doi: 10.1385/1-59259-789-0:077.

I/we claim:

1. A recombinant Modified Vaccinia Ankara (rMVA) vector comprising:
(i) a first expression cassette comprising a single nucleic acid transcript encoding three EBV glycoproteins, wherein the EBV glycoproteins are gp42, gL, and gH, and wherein the nucleic acid transcript further comprises a self-cleaving 2A peptide between each of the EBV glycoproteins; and
(ii) a second expression cassette comprising a single nucleic acid transcript encoding two EBV glycoproteins, wherein the EBV glycoproteins are gp350 and gB, and wherein the nucleic acid transcript further comprises a self-cleaving 2A peptide between each of the EBV glycoproteins.

2. The rMVA of claim 1, wherein the first expression cassette is inserted into an IGR3 insertion site.

3. The rMVA of claim 1, wherein the second expression cassette is inserted into a G1L insertion site.

4. The rMVA vector of claims 1, wherein the rMVA vector is genetically and translationally stable in 10 viral passages and expresses each of the EBV glycoproteins.

5. The rMVA vector of claims 1, wherein each of the first expression construct and the second expression construct comprise a promoter.

6. The rMVA vector of claim 5, wherein the promoter is a modified H5 (mH5) promoter or any other promotor capable of promoting expression of the antigens in each expression construct.

7. The rMVA vector of claim 6, wherein the promoter is mH5.

8. A composition comprising the rMVA vector of claims 1.

9. A vaccine or immunogenic fragment comprising the rMVA vector of claim 1.

10. A method of preventing or treating an EBV infection or a condition associated with an EBV infection comprising administering to a subject in need thereof the rMVA of claim 1.

11. A method of preventing or treating an EBV infection or a condition associated with an EBV infection comprising administering to a subject in need thereof the vaccine of claim 9.

12. The method of claim 10, wherein the administering to the subject elicits an IgG response against the EBV glycoproteins.

13. The method of claim 12, wherein the IgG response comprises a response of an IgG1 subtype.

14. The method of claim 12, wherein the IgG response comprises a response of an IgG2a subtype.

15. The method of claim 10, wherein the administering to the subject elicits a Th1- and Th2-type immune response.

16. An immunization regimen comprising administering to a subject in need thereof one or more doses of a therapeutically effective amount of the rMVA of claim 1.

17. The immunization regimen of claim 16, wherein the administering to the subject elicits an IgG response against the EBV glycoproteins.

18. The immunization regimen of claim 17, wherein the IgG response comprises a response of an IgG1 subtype.

19. The immunization regimen of claim 17, wherein the IgG response comprises a response of an IgG2a subtype.

20. The immunization regimen of claims 16, wherein the administering to the subject elicits a Th1- and Th2-type immune response.
